SEQUENCE LISTING (44 sequences)

SEQ ID NO: 1 - DNA, 3990 bp, unidentified
  misc_feature 1..3990: note = Francisella novicida
  source 1..3990: mol_type = genomic DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataacggatccgaattcgagctcgaaaatctgtactttcaaggcgagctccgtcgacaagcttctgctctggaacaccaccatcatcatcactaa

SEQ ID NO: 2 - protein, 1329 aa, unidentified
  REGION 1..1329: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNGSEFELENLYFQGELRRQASALEHHHHHH

SEQ ID NO: 3 - protein, 1329 aa, synthetic construct
  REGION 1..1329: note = Double mutant - Nickase
  PEPTIDE 1..1329: note = Double mutant - Nickase
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFGGGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNGSEFELENLYFQGELRRQASALEHHHHHH

SEQ ID NO: 4 - protein, 1329 aa, synthetic construct
  REGION 1..1329: note = Double mutant - Non specific endonuclease
  PEPTIDE 1..1329: note = Double mutant - Non specific endonuclease
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDGGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFGRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNGSEFELENLYFQGELRRQASALEHHHHHH

SEQ ID NO: 5 - RNA, 19 nt, synthetic construct
  misc_feature 1..19: note = crRNA constant region
  misc_feature 1..19: note = crRNA constant region
  source 1..19: mol_type = other RNA
aatttctactgttgtagat

SEQ ID NO: 6 - DNA, 33 bp, synthetic construct
  misc_feature 1..33: note = t-strand
  misc_feature 1..33: note = t-strand
  source 1..33: mol_type = other DNA
atgcagtggccttattaaatgacttctctaacg

SEQ ID NO: 7 - DNA, 33 bp, synthetic construct
  misc_feature 1..33: note = nt-strand
  misc_feature 1..33: note = nt-strand
  source 1..33: mol_type = other DNA
cgttagagaagtcatttaataaggccactgcat

SEQ ID NO: 8 - DNA, 33 bp, synthetic construct; identical to SEQ ID NO: 6
  misc_feature 1..33: note = t-strand
  misc_feature 1: note = t-strand
  source 1..33: mol_type = other DNA

SEQ ID NO: 9 - DNA, 33 bp, synthetic construct; identical to SEQ ID NO: 7
  misc_feature 1..33: note = nt-strand
  misc_feature 1..33: note = nt-strand
  source 1..33: mol_type = other DNA

SEQ ID NO: 10 - RNA, 43 nt, synthetic construct
  misc_feature 1..43: note = crRNA
  misc_feature 1..43: note = crRNA
  source 1..43: mol_type = other RNA
aatttctactgttgtagatcaccgagtgcagccactcggcgct

SEQ ID NO: 11 - DNA, 39 bp, unidentified
  misc_feature 1..39: note = Francisella novicida
  misc_feature 1..39: note = DNA sequence of REC domain
  source 1..39: mol_type = genomic DNA
ttatttaagcaaattttaagtgatacagaatctaaatct

SEQ ID NO: 12 - protein, 13 aa, unidentified
  REGION 1..13: note = Francisella novicida
  REGION 1..13: note = MISC_FEATURE - amino acid sequence of REC domain
LFKQILSDTESKS

SEQ ID NO: 13 - DNA, 39 bp, synthetic construct
  misc_feature 1..39: note = Synthetic sequence
  misc_feature 1..39: note = mutated REC domain, DNA sequence
  source 1..39: mol_type = other DNA
ggtggtggagccggtgcaagtgctggaggatctggatct

SEQ ID NO: 14 - protein, 13 aa, synthetic construct
  REGION 1..13: note = Synthetic sequence
  REGION 1..13: note = MISC_FEATURE - mutated REC domain, amino acid sequence
GGGAGASAGGSGS

SEQ ID NO: 15 - DNA, 39 bp, unidentified
  misc_feature 1..39: note = Francisella novicida
  misc_feature 1..39: note = Lid domain, DNA sequence
  source 1..39: mol_type = genomic DNA
gaggatttaaattttggatttaaaagagggcgtttcaag

SEQ ID NO: 16 - protein, 13 aa, unidentified
  REGION 1..13: note = Francisella novicida
  REGION 1..13: note = MISC_FEATURE - Lid domain, amino acid sequence
EDLNFGFKRGRFK

SEQ ID NO: 17 - DNA, 39 bp, synthetic construct
  misc_feature 1..39: note = Synthetic sequence
  misc_feature 1..39: note = mutated Lid domain, DNA sequence
  source 1..39: mol_type = other DNA
ggtggtggagccggtggagcagctggagggggagccggt

SEQ ID NO: 18 - protein, 13 aa, synthetic construct
  REGION 1..13: note = Synthetic sequence
  REGION 1..13: note = MISC_FEATURE - Mutated Lid domain
GGGAGGAAGGGAG

SEQ ID NO: 19 - DNA, 36 bp, unidentified
  misc_feature 1..36: note = Francisella novicida
  misc_feature 1..36: note = finger domain
  source 1..36: mol_type = genomic DNA
aaaggtataaatgaatatataaatctatactcacag

SEQ ID NO: 20 - protein, 12 aa, unidentified
  REGION 1..12: note = Francisella novicida
  REGION 1..12: note = MISC_FEATURE - Finger domain
KGINEYINLYSQ

SEQ ID NO: 21 - DNA, 36 bp, synthetic construct
  misc_feature 1..36: note = Synthetic sequence
  misc_feature 1..36: note = Mutated finger domain
  source 1..36: mol_type = other DNA
ggtggtggagccggtgcagctggaggagcttcaggt

SEQ ID NO: 22 - protein, 12 aa, synthetic construct
  REGION 1..12: note = Synthetic sequence
  REGION 1..12: note = MISC_FEATURE - mutated finger domain
GGGAGAAGGASG

SEQ ID NO: 23 - DNA, 37 bp, synthetic construct
  misc_feature 1..37: note = Synthetic sequence
  misc_feature 1..33: note = labelled non-specific ssDNA
  source 1..37: mol_type = other DNA
agcatgcccaaattgcttacatatgtgttacgacggt

SEQ ID NO: 24 - protein, 1307 aa, unidentified
  REGION 1..1307: note = Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 25 - protein, 1230 aa, unidentified
  REGION 1..1230: note = Lachnospiraceae bacterium COE1
MHENNGKIADNFIGIYPVSKTLRFELKPVGKTQEYIEKHGILDEDLKRAGDYKSVKKIIDAYHKYFIDEALNGIQLDGLKNYYELYEKKRDNNEEKEFQKIQMSLRKQIVKRFSEHPQYKYLFKKELIKNVLPEFTKDNAEEQTLVKSFQEFTTYFEGFHQNRKNMYSDEEKSTAIAYRVVHQNLPKYIDNMRIFSMILNTDIRSDLTELFNNLKTKMDITIVEEYFAIDGFNKVVNQKGIDVYNTILGAFSTDDNTKIKGLNEYINLYNQKNKAKLPKLKPLFKQILSDRDKISFIPEQFDSDTEVLEAVDMFYNRLLQFVIENEGQITISKLLTNFSAYDLNKIYVKNDTTISAISNDLFDDWSYISKAVRENYDSENVDKNKRAAAYEEKKEKALSKIKMYSIEELNFFVKKYSCNECHIEGYFERRILEILDKMRYAYESCKILHDKGLINNISLCQDRQAISELKDFLDSIKEVQWLLKPLMIGQEQADKEEAFYTELLRIWEELEPITLLYNKVRNYVTKKPYTLEKVKLNFYKSTLLDGWDKNKEKDNLGIILLKDGQYYLGIMNRRNNKIADDAPLAKTDNVYRKMEYKLLTKVSANLPRIFLKDKYNPSEEMLEKYEKGTHLKGENFCIDDCRELIDFFKKGIKQYEDWGQFDFKFSDTESYDDISAFYKEVEHQGYKITFRDIDETYIDSLVNEGKLYLFQIYNKDFSPYSKGTKNLHTLYWEMLFSQQNLQNIVYKLNGNAEIFYRKASINQKDVVVHKADLPIKNKDPQNSKKESMFDYDIIKDKRFTCDKYQFHVPITMNFKALGENHFNRKVNRLIHDAENMHIIGIDRGERNLIYLCMIDMKGNIVKQISLNEIISYDKNKLEHKRNYHQLLKTREDENKSARQSWQTIHTIKELKEGYLSQVIHVITDLMVEYNAIVVLEDLNFGFKQGRQKFERQVYQKFEKMLIDKLNYLVDKSKGMDEDGGLLHAYQLTDEFKSFKQLGKQSGFLYYIPAWNTSKLDPTTGFVNLFYTKYESVEKSKEFINNFTSILYNQEREYFEFLFDYSAFTSKAEGSRLKWTVCSKGERVETYRNPKKNNEWDTQKIDLTFELKKLFNDYSISLLDGDLREQMGKIDKADFYKKFMKLFALIVQMRNSDEREDKLISPVLNKYGAFFETGKNERMPLDADANGAYNIARKGLWIIEKIKNTDVEQLDKVKLTISNKEWLQYAQEHIL

SEQ ID NO: 26 - protein, 1307 aa, Succiniclasticum ruminis
MGNFGEFTHKYQVSKTLRFELIPQGKTLENVAKYGIVDDDKRRSENYKKLKPVIDRIYKYFIDESLKNVSIDWQPLYEAIIAYRKEQTTANVVRLKEEQEACRKAIAAWFEGKVPDKGSKDLKEFNKTQSKLFKELFGKELFTESVTQLLPGLSLTEEEKELLASFNKFTSYFKGFYVNRKNVFSADDISTSIPHRLVQENFPKFMDNCEAYRRIVEEYPELKAKLEGTAQATGIFIGFKLDNIFKVSFYNHLLQQSQIDLYNQFLCGIAGEEGTMRVQGLNVTLNLAMKQDKVLGQKLKSMPHRFIPLYKQILSDRTTLSFIPEAFQNDEEVLLTVEEYRKSLEAERTTGAVSDIFNSLQAADLRHVYVNPAKLTAFSQMLFEDWSLCRESLRNWKLRSYGKAATKKVREEIESWLKESAISLDELQAALADGTLSVIINQKVQSVITTLEQELAKPLPKKLKTAEEKESLKSLLDSVQEACHSLEMFAVGENMDTDPCFYVPLREAMEAIQPIIPLYNKVRNFATQKPYSIEKFKLNFSNPILASGWDENRERQTCAILFRKGEKYYLGIYNAKVKPDFSIIKAVKGGNCFEKVVYRQFPDFSKMMPKCTTQLKEVQQHFASSSEDYVLYNKKFIKPLTITKEIYDLNNVLFDGKKKFQIDYLRKTKDEDGYYHALHTWINFAKEFVASYESTSIYDTSTVLSTEQYVKLNDFYGDLDNLFYRIKFESVSEETISEFVDEGKLFLFQIYNKDFAEGATGAPNLHTIYWKAVFDPENMKNVVVKLNGQAELFYRPKSAMDIVRHKVGEKLVNRRLKDGTSLTEELHEELYLYANGKLKKKLSEAAAAVLPQAVIYDVHHEIVKDRRFTEDKFFFHVPLTLNYKCDKNAVQFNASVQEYLKENPDTYIIGIDRGERNLIYAVVIDPQGNIVEQKSFNVINGFDYHNKLEQREKERNKARQDWTTVGKIKELKQGYLSLVVHEITSMMVKYNAIVVLENLNVGFKRIRSGIAEKAVYQQFEKMLINKLNYLMFKDVEGAKPGSVLNAYQLTDRFESFASMRNQTGFLFYIPAAFTSKIDPATGFVDPFCWSAIKTLDDKKTFISGFDTLKYDNVTGNFILHFEMKKNKDFQKKLEGFMPEWDIVVEANKDRRDAEGKTFISGKRIEFVRENNGHGHYEDYLPCKKLVEILRQYDILFEDGKDVLPLIMKNGDSKLIHEVFKVIRLSLQMRNSNAESGEDFISSPVENNEGICFDSRLGVETLPKDADANGAYHIALKGLLLLEKIRHDERKLGISNSEWLNHIQSLRG

SEQ ID NO: 27 - protein, 1231 aa, Butyrivibrio fibrisolvens
MYYESLTKLYPIKKTIRNELVPIGKTLENIKKNNILEADEDRKIAYIRVKAIMDDYHKRLINEALSGFALIDLDKAANLYLSRSKSADDIESFSRFQDKLRKAIAKRLREHENFGKIGNKDIIPLLQKLSENEDDYNALESFKNFYTYFESYNDVRLNLYSDKEKSSTVAYRLINENLPRFLDNIRAYDAVQKAGITSEELSSEAQDGLFLVNTFNNVLIQDGINTYNEDIGKLNVAINLYNQKNASVQGFRKVPKMKVLYKQILSDREESFIDEFESDTELLDSLESHYANLAKYFGSNKVQLLFTALRESKGVNVYVKNDIAKTSFSNVVFGSWSRIDELINGEYDDNNNRKKDEKYYDKRQKELKKNKSYTIEKIITLSTEDVDVIGKYIEKLESDIDDIRFKGKNFYEAVLCGHDRSKKLSKNKGAVEAIKGYLDSVKDFERDLKLINGSGQELEKNLVVYGEQEAVLSELSGIDSLYNMTRNYLTKKPFSTEKIKLNFNKPTFLDGWDYGNEEAYLGFFMIKEGNYFLAVMDANWNKEFRNIPSVDKSDCYKKVIYKQISSPEKSIQNLMVIDGKTVKKNGRKEKEGIHSGENLILEELKNTYLPKKINDIRKRRSYLNGDTFSKKDLTEFIGYYKQRVIEYYNGYSFYFKSDDDYASFKEFQEDVGRQAYQISYVDVPVSFVDDLINSGKLYLFRVYNKDFSEYSKGRLNLHTLYFKMLFDERNLKNVVYKLNGQAEVFYRPSSIKKEELIVHRAGEEIKNKNPKRAAQKPTRRLDYDIVKDRRYSQDKFMLHTSIIMNFGAEENVSFNDIVNGVLRNEDKVNVIGIDRGERNLLYVVVIDPEGKILEQRSLNCITDSNLDIETDYHRLLDEKESDRKIARRDWTTIENIKELKAGYLSQVVHIVAELVLKYNAIICLEDLNFGFKRGRQKVEKQVYQKFEKMLIDKLNYLVMDKSREQLSPEKISGALNALQLTPDFKSFKVLGKQTGIIYYVPAYLTSKIDPMTGFANLFYVKYENVDKAKEFFSKFDSIKYNKDGKNWNTKGYFEFAFDYKKFTDRAYGRVSEWTVCTVGERIIKFKNKEKNNSYDDKVIDLTNSLKELFDSYKVTYESEVDLKDAILAIDDPAFYRDLTRRLQQTLQMRNSSCDGSRDYIISPVKNSKGEFFCSDNNDDTTPNDADANGAFNIARKGLWVLNEIRNSEEGSKINLAMSNAQWLEYAQDNTI

SEQ ID NO: 28 - DNA, 37 bp, synthetic construct; identical to SEQ ID NO: 23
  misc_feature 1..37: note = Synthetic sequence
  misc_feature 1..37: note = non-specific ssDNA
  source 1..37: mol_type = other DNA

SEQ ID NO: 29 - DNA, 3900 bp, synthetic construct
  misc_feature 1..3900: note = Synthetic sequence
  misc_feature 1..3900: note = FnCpf1 Q1025G mutant
  source 1..3900: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatggaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataac

SEQ ID NO: 30 - protein, 1287 aa, synthetic construct
  REGION 1..1287: note = Synthetic sequence
  REGION 1..1287: note = MISC_FEATURE - Q1025E Cpf1 mutant
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYGKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 31 - DNA, 3900 bp, synthetic construct
  misc_feature 1..3900: note = Synthetic sequence
  misc_feature 1..3900: note = Q1025G E1028G Cpf1 mutant
  source 1..3900: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatggaaagttaggaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataac

SEQ ID NO: 32 - protein, 1287 aa, synthetic construct
  REGION 1..1287: note = Synthetic sequence
  REGION 1..1287: note = MISC_FEATURE - Q1025G E1028G mutant
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYGKLGKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 33 - DNA, 3861 bp, synthetic construct
  misc_feature 1..3861: note = Synthetic sequence
  misc_feature 1..3861: note = Rec-deletion Cpf1 mutant
  source 1..3861: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgtttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatggaaagttaggaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataac

SEQ ID NO: 34 - protein, 1287 aa, synthetic construct; identical to SEQ ID NO: 32
  REGION 1..1287: note = Synthetic sequence
  REGION 1..1287: note = MISC_FEATURE - Rec-deletion Cpf1 mutant

SEQ ID NO: 35 - DNA, 3903 bp, synthetic construct
  misc_feature 1..3903: note = Synthetic sequence
  misc_feature 1..3903: note = Rec-substitution Cpf1 mutant
  source 1..3903: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttggtggtggagccggtgcaagtgctggaggatctggatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 36 - protein, 1300 aa, synthetic construct
  REGION 1..1300: note = Synthetic sequence
  REGION 1..1300: note = MISC_FEATURE - Rec-substitution Cpf1 mutant
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVGGGAGASAGGSGSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 37 - DNA, 3903 bp, synthetic construct
  misc_feature 1..3903: note = Synthetic sequence
  misc_feature 1..3903: note = LID-substitution Cpf1 mutant
  source 1..3903: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttggtggtggagccggtggagcagctggagggggagccggtgtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 38 - protein, 1300 aa, synthetic construct
  REGION 1..1300: note = Synthetic sequence
  REGION 1..1300: note = MISC_FEATURE - LID-substitution Cpf1 mutant
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFGGGAGGAAGGGAGVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 39 - DNA, 3814 bp, synthetic construct
  misc_feature 1..3814: note = Synthetic sequence
  source 1..3814: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttattagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 40 - protein, 1287 aa, synthetic construct
  REGION 1..1287: note = Synthetic sequence
  REGION 1..1287: note = MISC_FEATURE - LID-deletion Cpf1 mutant
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 41 - DNA, 3867 bp, synthetic construct
  misc_feature 1..3867: note = Synthetic sequence
  misc_feature 1..3867: note = Finger-deletion Cpf1 mutant
  source 1..3867: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagacaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 42 - protein, 1288 aa, synthetic construct
  REGION 1..1288: note = Synthetic sequence
  REGION 1..1288: note = MISC_FEATURE - Finger-deletion Cpf1 mutant
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 43 - protein, 1238 aa, unidentified
  REGION 1..1238: note = Candidatus Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 44 - protein, 1272 aa, unidentified
  REGION 1..1272: note = uncultured Clostridium sp.
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKCLEKIQEKKRKEIYQYLSDDAVFSEMFKEKMISGILPDFIRCNEEYSEEEKEEKLKTVALFHRFTSSFNDFFLNRKNVFTKEAIATAIGYRVVHENAEIFLENMVAFQNIQKSAESQISIIERKNEHYFMEWKLSHIFTADYYMMLMTQKAIEHYNEMCGVVNQHMKEYCQKEKKNWNLYRMKRLHKQILSNASTSFKIPEKYENDAEVYESVNSFLQNVMEKTVMERIAVLKNNTDNFDLSKIYITAPYYEKISNYLCGSWNTIADCLTHYYEQQIAGKGARKDQKVKAAVKADKWKSLSEIEQLLKEYARAEEVKRKPEEYIAEIENIVSLKEVHLLEYHPEVNLIENEKYATEIKDVLDNYMELFHWMKWFYIEEAVEKEVNFYGELDDLYEEIRDIVPLYNKVRNYVTQKPYSDTKIKLNFGTPTLANGWSKSKEYDYNAILLQKDGKYYMGIFNPVQKPEKEIIEGHSHPLEGNEYKKMVYYYLPSANKMLPKVLLSKKGMEIYQPSEYIINGYKERRHIKSEEKFDLQFCHDLIDYFKSGIERNPDWKVFGFHFSDTDTYQDISGFYREVEDQGYKIDWTYIKEADIDRLNEEGKLYLFQIYNKDFSEKSTGRENLHTMYLKNLFSEENIREQVLKLNGEAEIFFRKSSVKKPIIHKKGTMLVNRTYMEEMHGESVKKNIPEKEYQEIYNYMNHRWKGELSAEAKEYLKKAVCHETKKDIVKDYRYSVDKFFIHLPITINYRASGKEALNSVAQRYIAHQNDMHVIGIDRGERNLIYVSVINMQGEIIEQKSFNVVNKYNYKEKLKEREQNRDEARKNWKEIGQIKDLKEGYLSGVIHEIAKMMIKYHAIVAMEDLNYGFKRGRFKVERQVYQKFENMLIQKLNYLVFKDRSADEDGGVLRGYQLAYIPDSVKKLGRQCGMIFYVPAAFTSKIDPATGFVDIFNHKAYTTDQAKREFILSFDEICYDVERQLFRFTFDYANFATHNVTLARNNWTIYTNGTRTQKEFVNRRVRDKKEVFDPTEKMLKLLELEGVEYQSGANLLPKLEKISDPHLFHELQRIVRFTVQLRNSKNEENDVDYDHVISPVLNEEGKFFDSSKYENKEEKKESLLPVDADANGAYCIALKGLYIMQAIQKNWSEEKALSPDVLRLNNNDWFDYIQNKRYR

(19) United States
(12) Patent Application Publication
(10) Pub. No.: US 2025/0163394 A1
(43) Pub. Date: May 22, 2025

(54) Mutant Cpf1 Endonucleases

(71) Applicant: University of Copenhagen, Copenhagen K (DK)

(72) Inventors: Stefano STELLA, Boston, MA (US); Guillermo MONTOYA, Frederiksberg (DK)

(73) Assignee: University of Copenhagen, Copenhagen K (DK)

(21) Appl. No.: 18/925,815

(22) Filed: Oct. 24, 2024

Related U.S. Application Data
(62) Division of application No. 15/734,839, filed on Dec. 3, 2020, now Pat. No. 12,152,257.

(30) Foreign Application Priority Data
Jun. 4, 2018 (EP) 18175707.1
Aug. 27, 2018 (EP) 18190950.8

(51) Int. Cl. C12N 9/22; C12Q 1/6806

(52) U.S. Cl. CPC C12N 9/22; C12Q 1/6806

(57) ABSTRACT

The present invention relates to mutant Cpf1 endonucleases having altered activity compared to the wild type Cpf1, and their use to introduce single strand breaks in nucleic acid sequences. Methods for detection and quantification of a nucleic acid sequence are also disclosed. Methods for diagnosis of an infectious disease are also disclosed.

### CROSS REFERENCE TO RELATED APPLICATIONS

This application is a divisional of U.S. application Ser. No. 15/734,839, filed Dec. 3, 2020, which is a national stage application under 35 U.S.C. § 371 of International Patent Application No. PCT/EP2019/064444, filed Jun. 4, 2019, which claims priority to and the benefit of EP Application No. 18175707.1, filed Jun. 4, 2018, and EP Application No. 18190950.8, filed Aug. 27, 2018. All of the aforementioned priority applications are incorporated herein by reference in their entirety.

### SEQUENCE LISTING

The XML file submitted herewith via Patent Center, entitled “125665.D1003.xml,” created on Nov. 25, 2024, and having a size of 120,858 bytes, is incorporated herein by reference in its entirety.

### TECHNICAL FIELD

The present invention relates to mutant Cpf1 (also known as Cas12a) endonucleases having altered activity compared to the wild type Cpf1, and their use to both introduce single strand breaks or double strand breaks in nucleic acid target sequences which are either single stranded or double stranded and single strand DNA breaks upon binding (but not cleavage) on the DNA-target either single stranded or double stranded non-specific manner. Methods for detection and quantification of a nucleic acid sequence are also disclosed. Methods for diagnosis of an infectious disease are also disclosed.

### BACKGROUND

Cpf1 is a DNA endonuclease, belonging to the CRISPR-Cas (Clustered regularly interspaced short palindromic repeats) class 2 type V-B adaptive immune system, that the bacteria and archaea use to protect the cells against the invasion of potential dangerous DNA molecules (Makarova et al., 2011; Makarova and Koonin, 2015; Vestergaard et al., 2014). Cpf1 forms with a relatively small (between 40 to 45 bases) crRNA (CRISPR-RNA) molecule, a ribonucleoprotein complex that is able to recognize, unwind and cut with high specificity the DNA-target complementary to the crRNA. The Cpf1 DNA-target sequences are stored in the CRISPR-array that is a region of DNA containing repetitive sequences adjacent to unique sequences (spacers) that are the record of the potential dangerous DNA previously encountered by the cell (Zetsche et al., 2015). The CRISPR array is transcribed as single pre-crRNA that is then processed by Cpf1 to produce mature crRNAs (Fonfara et al., 2016). These RNAs thus contain a conserved part, identical for all the crRNAs, which folds in a pseudoknot structure recognized by Cpf1, and a variable region that is complementary to the target DNAs. To maintain the integrity of the CRISPR-array the cleavage of the DNA-target is bound to the recognition of a short PAM sequence (protospacer adjacent motif) usually 3 or 4 nucleotides length upstream of the target site. The PAM sequence that is recognized by Cpf1 is present in the invading DNA molecules but not in the CRISPR-array thus protecting this DNA region from cleavage (Mojica et al., 2005).

Some CRISPR endonucleases such as Cas9, Cpf1, and Cas13 have been used to modify the genomes of several organisms as well as diagnostic molecules to identify infections and also as potential tools for gene therapy (Chen et al., 2018; Gootenberg, 2018).

However, CRISPR endonucleases have so far being used in the same way as they act in nature. There is a need to discover the full potential of these enzymes and optimize them for use in known as well as new applications.

### SUMMARY

The present disclosure relates to mutant Cpf1 endonucleases that are capable of introducing single strand breaks or double strand breaks in nucleic acid target sequences which are either single stranded or double stranded. Furthermore, mutant Cpf1 endonucleases of the present disclosure are able to bind (without cutting) the DNA-target either single stranded or double stranded, and to cleave in a non-specific manner single stranded DNA. Moreover, mutant Cpf1 endonucleases, and the complexes they form with crRNAs, capable of introducing one or more single strand or double strand breaks in a nucleic acid sequence, which is different from the nucleic acid sequence recognized and hybridized by the crRNA are disclosed.

The new mutant Cpf1 endonucleases disclosed herein present several advantages over wild type Cpf1 endonucleases and can be favourably used for detection and quantification of target nucleic acid sequences in a specific or non-specific manner, even at very low concentrations, such as at concentrations below the picomolar range.

The new mutant Cpf1 endonucleases disclosed herein present several advantages over wild type Cpf1 endonucleases and can be favourably used for cutting target nucleic acid sequences both double stranded and single stranded. Furthermore, in the double stranded target the cut can be on both strands or on one strand only.

Finally, the new mutant Cpf1 endonucleases disclosed herein may also be used for diagnosis of an infectious disease, by detection of genetic material deriving from the infectious agent causing the disease.

One aspect of the present disclosure relates to a mutant Cpf1 endonuclease or orthologue thereof comprising a polypeptide sequence having at least 95% sequence identity to:
i) the sequence corresponding to residues 1 to 323, 337 to 1005, and 1019 to 1329 of SEQ ID NO: 2, wherein said polypeptide sequence further comprises:
   a. at least two amino acid mutations in, the REC domain (residues 324 to 336; SEQ ID NO: 16) compared to SEQ ID NO: 2, wherein each mutation independently is an amino acid substitution or deletion; and/or
   b. at least two amino acid substitutions in the Lid domain (residues 1006 to 1018; SEQ ID NO: 20) compared to SEQ ID NO: 2, wherein at least one of the residues at positions 917 and 1006 is a glutamic acid (E) or an aspartic acid (D);
      and/or
ii) SEQ ID NO: 2, wherein said polypeptide sequence comprises at least two amino acid substitutions in two positions independently selected from the positions corresponding to residues 918, 1013, 1014, 1025 and 1028 of SEQ ID NO: 2.

Another aspect of the present disclosure relates to a polynucleotide encoding the mutant Cpf1 endonuclease or orthologue thereof as disclosed herein.

Another aspect of the present disclosure relates to a recombinant vector comprising a polynucleotide or a nucleic acid sequence encoding a mutant Cpf1 endonuclease or orthologue thereof as disclosed herein, said polynucleotide or nucleic acid sequence being optionally linked to a promoter.

A further aspect of the present disclosure relates to a cell expressing the mutant Cpf1 or orthologue thereof, or the polynucleotide, nucleic acid or recombinant vector as disclosed herein.

An even further aspect of the present disclosure relates to a system for expression of a crRNA-Cpf1 complex comprising
a. a polynucleotide or a recombinant vector comprising a polynucleotide encoding a mutant Cpf1 endonuclease or orthologue thereof, or a nucleic acid as disclosed herein; and
b. a polynucleotide or a recombinant vector comprising a polynucleotide encoding a guide RNA (crRNA) operably linked to a promoter.

Another aspect of the present disclosure relates to a use of a crRNA-Cpf1 complex for introducing a single strand break in a first target nucleic acid, wherein:
a. a Cpf1 endonuclease or orthologue thereof is contacted with a guide RNA (crRNA), thereby obtaining a crRNA-Cpf1 complex capable of recognising a second target nucleic acid, the second target nucleic acid comprising a protospacer adjacent motif (PAM), and
b. the crRNA-Cpf1 complex is contacted with the first target nucleic acid;
whereby a single strand break is made in the first target sequence.

Another aspect of the present disclosure relates to a method of introducing a single strand break in a first target nucleic acid, comprising the steps of:
a. designing a guide-RNA (crRNA) capable of recognising a second target nucleic acid comprising a protospacer adjacent motif (PAM);
b. contacting the crRNA of step a. with a Cpf1 endonuclease or orthologue thereof, thereby obtaining a crRNA-Cpf1 complex capable of binding to said second target nucleic acid, and
c. contacting the crRNA and the Cpf1 with said first target nucleic acid, thereby introducing one or more single strand breaks in the first target nucleic acid.

A further aspect of the present disclosure relates to an in vitro method of introducing a site-specific, double-stranded break at a second target nucleic acid in a mammalian cell, the method comprising introducing into the mammalian cell a crRNA-Cpf1 complex, wherein the Cpf1 is a mutant Cpf1 endonuclease or orthologue as disclosed herein, and wherein the crRNA is specific for the second target nucleic acid.

A further aspect of the present disclosure relates to a method for detection of a second target nucleic acid in a sample, the method comprising:
a. Providing a crRNA-Cpf1 complex, wherein the Cpf1 is a Cpf1 endonuclease or orthologue thereof as disclosed herein, and wherein the crRNA is specific for the second target nucleic acid;
b. Providing a labelled ssDNA, wherein the ssDNA is labelled with at least one set of interactive labels comprising at least one dye and at least one quencher;
c. Contacting the crRNA-Cpf1 complex and the ssDNA with the sample, wherein the sample comprises at least one second target nucleic acid; and
d. Detecting cleavage of the ssDNA by detecting a fluorescent signal from the fluorophore,
thereby detecting the presence of the second target nucleic acid in the sample.

Another aspect of the present disclosure relates to an in vitro method for diagnosis of an infectious disease in a subject, the method comprising:
a. Providing a crRNA-Cpf1 complex, wherein the Cpf1 is a Cpf1 endonuclease or orthologue thereof as disclosed herein, and wherein the crRNA is specific for a second target nucleic acid;
b. Providing a labelled ssDNA, wherein the ssDNA is labelled with at least one set of interactive labels comprising at least one dye and at least one quencher;
c. Providing a sample from the subject, wherein said sample comprises or is suspected of comprising the second target nucleic acid; and
d. Determining the level and/or concentration of the second target nucleic acid according to the methods disclosed herein,
   wherein the second target nucleic acid is a nucleic acid of the genome of an infectious agent causing the disease or a fragment thereof,
   thereby diagnosing an infectious disease in a subject.

### DESCRIPTION OF THE DRAWINGS

FIG. 1. Representation of the crRNA and the double strand DNA target.

FIG. 2. Cleavage products of the double strand DNA target obtained by using wild type Cpf1 and mutant 1 (SEQ ID NO: 3).

FIG. 3. Left panel: cleavage products of the double strand DNA target obtained by using wild type Cpf1 and mutant 1 (SEQ ID NO: 4, 918-1013 mutant) (black circles). Right panel: cleavage products of the non-specific ssDNA obtained by using wild type Cpf1 and mutant 2 (SEQ ID NO: 4, 918-1013 mutant) (black circle).

FIG. 4. (A) Cleavage activity assays with target DNA substrate for the REC-linker deletion and substitution (SEQ ID NO: 34 and 36), the lid substitution (SEQ ID NO: 38) and the Q1025G, E1028G and Q1025G-E1028G mutants (SEQ ID NO: 30 and 32). (B) Cleavage activity assays monitoring the cut of the t-strand complementary to crRNA for the REC-linker deletion and substitution, the lid substitution and the Q1025G, E1028G and Q1025G-E1028G mutants. (C) Cleavage activity assays upon activation with ssDNA complementary to crRNA using non-specific ssDNA as substrate for the REC-linker deletion and substitution, the lid substitution and the Q1025G, E1028G and Q1025G-E1028G mutants. (D) Cleavage activity assays monitoring the cut of the dsDNA substrate (t-strand, nt-strand and non-specific ssDNA for wild type Cpf1, (D917)Cpf1, (E1006-D917A)Cpf1 and (Lid substitution-D917A)Cpf1.

FIG. 5 Protein expression and purification profiles mutant Cpf1 with deletions or substitutions in the REC-linker, lid and finger regions.

FIG. 6. A. Quantification of the cleavage activities: NTS (Non-Target Strand), TS (Target Strand) and ssDNA (non-specific single strand) of Cas12a wild type (cas12aWT); B. Cpf1 Q1025G (SEQ ID NO: 30); C. Cpf1 Q1025G-E1028G (SEQ ID NO: 32); D. nickase Cpf1 K1013G-R1014G (SEQ ID NO: 3). Each bar represents the average of at least 3 independent replicates and the standard deviation is shown.

### DETAILED DESCRIPTION

The invention is as defined in the claims.

The present disclosure relates to mutant Cpf1 endonucleases or orthologues thereof and their use to introduce single strand breaks or double strand breaks in nucleic acid target sequences which are either single stranded or double stranded in a specific or non-specific manner. Throughout the present disclosure a “mutant Cpf1 endonuclease” may be a naturally occurring mutant, for example a mutant encoded by a Cpf1 gene carrying one or more single nucleotide polymorphisms (SNPs), or a non-naturally occurring mutant, for example a mutant obtained by direct mutagenesis or random mutagenesis of the Cpf1 gene.

The inventors have identified mutants of Cpf1 with modified activities. Some of the mutants disclosed herein are able to function as a nickase, thus recognizing and introducing a single strand break at a specific target DNA sequence. The inventors have also identified mutants of Cpf1 that recognize a target DNA sequence and, following binding to said target nucleic acid, introduce a single strand break at non-target DNA sequences. Some mutants can cleave the DNA in a single stranded sequence, others in a double stranded sequence. The cleavage may be specific or non-specific. The cleavage may be activated by double stranded target DNA, by single stranded target DNA or both.

The inventors also provide methods for detecting and quantifying a target DNA sequence using a mutant Cpf1 endonuclease. The inventors also provide methods for diagnosing an infectious disease by detecting and quantifying a target DNA sequence using a mutant Cpf1 endonuclease.

### Definitions

Codon The term “codon” as used herein refers to a triplet of adjacent nucleotides coding for a specific amino acid.

CRISPR-Cas system The term refers to members of the CRISPR-Cas family. The prokaryotic adaptive immune system CRISPR-Cas (clustered regularly interspaced short palindromic repeats and CRISPR-associated proteins) can bind and cleave a target DNA sequence through RNA-guided recognition. According to their molecular architecture, the different members of the CRISPR-Cas system have been classified in two classes: class 1 encompasses several effector proteins, whereas class 2 systems use a single element (Makarova et al., 2015). Cpf1 (“CRISPR from Prevotella and Francisella”) has been described as a new member of class 2 type V CRISPR-Cas endonucleases present in a number of bacterial genomes (Zetsche et al., 2015).

Endonuclease The term refers to an enzyme capable of cleaving the phosphodiester bond within a polynucleotide chain. Some endonucleases are specific, i.e. they recognise a given nucleotide sequence which directs the site of cleavage, some are non-specific. The present disclosure is directed to both specific and non-specific endonucleases. One example of endonucleases is nicking endonucleases. A nicking endonuclease as used herein is referred to an enzyme that cuts one strand of a double-stranded DNA to produce a “nicked” DNA molecule. A nicking endonuclease as used herein refers also to an endonuclease that cuts one strand of a single stranded DNA.

Fragment The term is used to indicate a non full-length part of a nucleic acid or polypeptide. Thus, a fragment is itself also a nucleic acid or polypeptide, respectively. DNA fragments are designated starting from the 5′-end throughout the present disclosure.

Gene editing The term refers to the use of genetic engineering procedures to insert, delete or replace one or more nucleotides in a nucleotide sequence.

Guide RNA The term will herein be used interchangeably with “crRNA” and refers to the RNA molecule which is required for recognition of a target nucleic acid sequence by CRISPR-Cas proteins, in particular Cpf1.

Homologue A homologue or functional homologue may be any polypeptide that exhibits at least some sequence identity with a reference polypeptide and has retained at least one aspect of the original functionality. Herein a functional homologue of Cpf1 is a polypeptide sharing at least some sequence identity with Cpf1 or a fragment thereof which has the capability to function as an endonuclease similarly to Cpf1, i.e. it is capable of specifically binding a crRNA, and of specifically recognising, binding and cleaving a target nucleic acid.

Protospacer adjacent motif (PAM) The term refers to the DNA sequence immediately downstream the DNA sequence targeted by a CRISPR-Cas system such as Cpf1. The crRNA of a crRNA-Cpf1 complex is capable of recognizing and hybridizing only a target DNA sequence comprising a PAM.

Recognition The term “recognition” as understood herein refers to the ability of a molecule to identify a nucleotide sequence. For example, an enzyme or a DNA binding domain may recognise a nucleic acid sequence as a potential substrate and bind to it. Preferably, the recognition is specific.

Sequence identity As used herein, the term refers to two polynucleotide sequences that are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polypeptides, peptides or proteins, a degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences.

The global percentage of sequence identity is determined with the algorithm GAP, BESTFIT, or FASTA in the Wisconsin Genetics Software Package Release 7.0, using default gap weights.

The term “interactive labels” or “set of interactive labels” as used herein refers to at least one fluorophore and at least one quencher which can interact when they are located adjacently. When the interactive labels are located adjacently the quencher can quench the fluorophore signal. The interaction may be mediated by fluorescence resonance energy transfer (FRET).

The term “located adjacently” as used herein refers to the physical distance between two objects. If a fluorophore and a quencher are located adjacently, the quencher is able to partly or fully quench the fluorophore signal. FRET quenching may typically occur over distances up to about 100 Å. Located adjacently as used herein may refer to distances below and/or around 100 Å.

The term “fluorescent label” or “fluorophore” as used herein refers to a fluorescent chemical compound that can re-emit light upon light excitation. The fluorophore absorbs light energy of a specific wavelength and re-emits light at a longer wavelength. The absorbed wavelengths, energy transfer efficiency, and time before emission depend on both the fluorophore structure and its chemical environment, as the molecule in its excited state interacts with surrounding molecules. Wavelengths of maximum absorption (˜ excitation) and emission (for example, Absorption/Emission=485 nm/517 nm) are the typical terms used to refer to a given fluorophore, but the whole spectrum may be important to consider.

The term “quench” or “quenching” as used herein refers to any process which decreases the fluorescence intensity of a given substance such as a fluorophore. Quenching may be mediated by fluorescence resonance energy transfer (FRET). FRET is based on classical dipole-dipole interactions between the transition dipoles of the donor (e.g. fluorophore) and acceptor (e.g. quencher) and is dependent on the donor-acceptor distance. FRET can typically occur over distances up to 100 Å. FRET also depends on the donor-acceptor spectral overlap and the relative orientation of the donor and acceptor transition dipole moments. Quenching of a fluorophore can also occur as a result of the formation of a non-fluorescent complex between a fluorophore and another fluorophore or non-fluorescent molecule. This mechanism is known as ‘contact quenching,’ ‘static quenching,’ or ‘ground-state complex formation

The term “quencher” as used herein refers to a chemical compound which is able to quench a given substance such as a fluorophore.

Target strand and non-target strand The target strand refers to the nucleic acid strand which interacts with the crRNA to form a crRNA-DNA hybrid. The non-target strand is complementary to the target strand, and is displaced to the RuvC/NuC pocket of the Cpf1 endonuclease.

The term “orthologue” as used herein refers to genes (and proteins encoded by said genes) inferred to be descended from the same ancestral sequence separated by a speciation event: when a species diverges into two separate species, the copies of a single gene in the two resulting species are said to be orthologous. Orthologs, or orthologous genes, are genes in different species that originated by vertical descent from a single gene of the last common ancestor. Cpf1 orthologues can be identified and characterized based on sequence similarities to the present system, as has been described with type II systems for example. For example, orthologues of Cpf1 include F. novicida U1 12, Prevotella albensis, Acidaminococcus sp. BV3L6, Eubacterium eligens CAG: 72, Butyrivibrio fibrisolvens, Smithella sp. SCADC, Flavobacterium sp. 316, Porphyromonas crevioricanis, Bacteroidetes oral taxon 274, and Lachnospiraceae bacterium ND2006.

### Mutant Cpf1

The inventors have identified three domains of Cpf1 which appear to be involved in different activities of the enzyme. These three domains are:
The REC domain
The Lid domain
The finger domain.

In FnCpf1 (SEQ ID NO: 2), these three domains are defined as follows:
REC domain: from residue 324 to residue 336;
Lid domain: from residue 1006 to 1018;
Finger domain: from residue 298 to 309.

Substitution or deletion of amino acids in any of these three domains results in modified enzyme activity, as will be detailed herein below. In addition, five residues were identified which appear important for enzymatic activity, i.e. mutations or deletions of any of these three residues also modifies enzyme activity. These residues are at position 918, 1013, 1014, 1025 and 1028, of which 1013 and 1014 are part of the Lid region. The present disclosure thus relates to modified Cpf1 proteins having altered activities.

One aspect of the present disclosure thus relates to a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to:
i) the sequence corresponding to residues 1 to 297, 310 to 323, 337 to 1005, and 1019 to 1329 of SEQ ID NO: 2, wherein said polypeptide sequence further comprises at least one amino acid substitution or deletion in the finger domain (residues 298 to 309; SEQ ID NO: 12), the REC domain (residues 324 to 336; SEQ ID NO: 16) or the Lid domain (residues 1006 to 1018; SEQ ID NO: 20) compared to SEQ ID NO: 2; and/or
ii) SEQ ID NO: 2, wherein said polypeptide sequence comprises at least one amino acid substitution in position 918, 1013, 1014, 1025 or 1028 of SEQ ID NO: 2.

One aspect of the present disclosure thus relates to a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to:
i) the sequence corresponding to residues 1 to 323, 337 to 1005, and 1019 to 1329 of SEQ ID NO: 2, wherein said polypeptide sequence further comprises:
   a. at least two amino acid mutations in the REC domain (residues 324 to 336; SEQ ID NO: 16) compared to SEQ ID NO: 2, wherein each mutation independently is an amino acid substitution or deletion; and/or
   b. at least two amino acid substitutions in the Lid domain (residues 1006 to 1018; SEQ ID NO: 20) compared to SEQ ID NO: 2, wherein at least one of the residues at positions 917 and 1006 is a glutamic acid (E) or an aspartic acid (D);
      and/or
ii) SEQ ID NO: 2, wherein said polypeptide sequence comprises at least two amino acid substitutions in two positions independently selected from the positions corresponding to residues position 918, 1013, 1014, 1025 and 1028 of SEQ ID NO: 2.

One aspect of the present disclosure relates to a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least one amino acid substitution in position 918, 1013, or 1014 of SEQ ID NO: 2.

One aspect of the present disclosure relates to a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions in two positions independently selected from the positions corresponding to residues 918, 1013, 1014, 1025 and 1028 of SEQ ID NO: 2.

In one embodiment the Cpf1 endonuclease is derived from Francisella novicida.

In some embodiments, the mutant Cpf1 comprises a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% identity to the sequence corresponding to residues 1 to 323 and 337 to 1329 of SEQ ID NO: 2, wherein said polypeptide sequence further comprises at least one amino acid substitution or deletion in the finger domain (residues 298 to 309; SEQ ID NO: 12), the REC domain (residues 324 to 336; SEQ ID NO: 16) or Lid finger domain (residues 1006 to 1018; SEQ ID NO: 20) compared to SEQ ID NO: 2. The mutant Cpf1 may further comprise at least one amino acid substitution in position 918, 1013, 1014, 1025 or 1028 of SEQ ID NO: 2.

In some embodiments, the mutant Cpf1 comprises a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% identity to the sequence corresponding to residues 1 to 323 and 337 to 1329 of SEQ ID NO: 2, wherein said polypeptide sequence further comprises at least two mutations in the REC domain (residues 324 to 336; SEQ ID NO: 16), wherein each mutation independently is an amino acid substitution or deletion, or at least two amino acid substitutions in the Lid domain (residues 1006 to 1018; SEQ ID NO: 20), compared to SEQ ID NO: 2. The mutant Cpf1 comprising mutations in the REC and/or Lid domain may further comprise at least one amino acid substitution in position 918, 1013, 1014, 1025 or 1028 of SEQ ID NO: 2.

In one embodiment, the mutant Cpf1 has at least one amino acid substitution or deletion in the finger domain. The at least one amino acid substitution or deletion may be substitution or deletion of 1, 2, 3, 4, 5, 6, 7, 8, 9, 10, 11 or 12 contiguous or non-contiguous amino acids of the finger domain defined as residues 298 to 309 of SEQ ID NO: 2. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 297 and 310 to 1329 of SEQ ID NO: 2. The mutant Cpf1 may further comprise at least one amino acid substitution in position 918, 1013, 1014, 1025 or 1028 of SEQ ID NO: 2.

In another embodiment, the mutant Cpf1 has at least one amino acid substitution or deletion in the REC domain. The at least one amino acid substitution or deletion may be substitution or deletion of 1, 2, 3, 4, 5, 6, 7, 8, 9, 10, 11, 12 or 13 contiguous or non-contiguous amino acids of the REC domain defined as residues 324 to 336 of SEQ ID NO: 2. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 323 and 337 to 1329 of SEQ ID NO: 2. The mutant Cpf1 may further comprise at least one amino acid substitution in position 918, 1013, 1014, 1025 or 1028 of SEQ ID NO: 2.

In another embodiment, the mutant Cpf1 has at least two amino acid mutations in the REC domain, wherein each mutation independently is an amino acid substitution or deletion. The at least two amino acid mutations may be substitutions and/or deletions of 1, 2, 3, 4, 5, 6, 7, 8, 9, 10, 11, 12 or 13 contiguous or non-contiguous amino acids of the REC domain defined as residues 324 to 336 of SEQ ID NO: 2. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 323 and 337 to 1329 of SEQ ID NO: 2. The mutant Cpf1 comprising at least two mutations in the REC domain may further comprise at least one amino acid substitution in position 918, 1013, 1014, 1025 or 1028 of SEQ ID NO: 2.

In another embodiment, the mutant Cpf1 has at least one amino acid substitution or deletion in the lid domain. The at least one amino acid substitution or deletion may be substitution or deletion of 1, 2, 3, 4, 5, 6, 7, 8, 9, 10, 11, 12 or 13 contiguous or non-contiguous amino acids of the Lid domain defined as residues 1006 to 1013 of SEQ ID NO: 2. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 1005 and 1019 to 1329 of SEQ ID NO: 2. The mutant Cpf1 may further comprise at least one amino acid substitution in position 918, 1013, 1014, 1025 or 1028 of SEQ ID NO: 2.

In another embodiment, the mutant Cpf1 has at least two amino acid substitutions in the Lid domain. The at least two amino acid substitutions may be substitutions of 1, 2, 3, 4, 5, 6, 7, 8, 9, 10, 11, 12 or 13 contiguous or non-contiguous amino acids of the Lid domain defined as residues 1006 to 1013 of SEQ ID NO: 2. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 1005 and 1019 to 1329 of SEQ ID NO: 2. The mutant Cpf1 comprising at least two substitutions in the Lid domain may further comprise at least one amino acid substitution in position 918, 1013, 1014, 1025 or 1028 of SEQ ID NO: 2

In another embodiment, Cpf1 has at least one amino acid substitution or deletion in the finger domain and in the REC domain, where the at least one amino acid substitution or deletion is as defined above. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 297, 310 to 323 and 337 to 1329 of SEQ ID NO: 2. The mutant Cpf1 may further comprise at least one amino acid substitution in position 918, 1013, 1014, 1025 or 1028 of SEQ ID NO: 2.

In another embodiment, Cpf1 has at least two amino acid mutations in the Lid domain and in the REC domain, wherein each mutation independently is an amino acid substitution or deletion as defined above. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 297, 310 to 323 and 337 to 1329 of SEQ ID NO: 2. The mutant Cpf1 comprising at least two amino acid mutations in the Lid domain and in the REC domain may further comprise at least one amino acid substitution in position 918, 1013, 1014, 1025 or 1028 of SEQ ID NO: 2.

In another embodiment, Cpf1 has at least one amino acid substitution or deletion in the finger domain and in the lid domain, where the at least one amino acid substitution or deletion is as defined above. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 297, 310 to 1005 and 1019 to 1329 of SEQ ID NO: 2. The mutant Cpf1 may further comprise at least one amino acid substitution in position 918, 1013, 1014, 1025 or 1028 of SEQ ID NO: 2.

In another embodiment, Cpf1 has at least one amino acid substitution or deletion in the REC domain and in the lid domain, where the at least one amino acid substitution or deletion is as defined above. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 323, 336 to 1005 and 1019 to 1329 of SEQ ID NO: 2. The mutant Cpf1 may further comprise at least one amino acid substitution in position 918, 1013, 1014, 1025 or 1028 of SEQ ID NO: 2.

In another embodiment, Cpf1 has at least one amino acid substitution or deletion in the finger domain, in the REC domain and in the lid domain, where the at least one amino acid substitution or deletion is as defined above. Accordingly, the mutant Cpf1 may comprise a sequence having at least 80%, such as at least 85%, at least 90%, at least 95%, at least 96%, at least 97%, at least 98%, at least 99% or 100% sequence identity to the sequence corresponding to residues 1 to 297, 310 to 323, 337 to 1005 and 1019 to 1329 of SEQ ID NO: 2. The mutant Cpf1 may further comprise at least one amino acid substitution in position 918, 1013, 1014, 1025 or 1028 of SEQ ID NO: 2.

In a specific embodiment, the Cpf1 mutant is as set forth in SEQ ID NO: 42, i.e. the finger domain is deleted. In another embodiment, the Cpf1 mutant is as set forth in SEQ ID NO: 34, i.e. the REC domain is deleted. In another embodiment, the Cpf1 mutant is as set forth in SEQ ID NO: 36, i.e. the REC domain is substituted. In another embodiment, the Cpf1 mutant is as set forth in SEQ ID NO: 40, i.e. the Lid domain is deleted. In another embodiment, the Cpf1 mutant is as set forth in SEQ ID NO: 38, i.e. the Lid domain is substituted.

It will be understood that the at least one amino acid substitution or deletion as defined above may refer to deletion of some amino acids in a domain, while other amino acids may be substituted.

All of the above mutants may further comprise at least one amino acid substitution and/or deletion in one or more of positions 918, 1013, 1014, 1025 or 1028 of SEQ ID NO: 2, as further detailed herein below. In one embodiment, the mutant further comprises at least one amino acid substitution at one of positions 918, 1013, 1014, 1025 or 1028 of SEQ ID NO: 2, the other positions remaining unmodified. In one embodiment, the mutant thus further comprises one substitution at one of positions 918, 1013, 1014, 1025 or 1028 of SEQ ID NO: 2. For example, the mutant further comprises one substitution at position 918, or at position 1013, or at position 1014, or at position 1025, or at position 1028.

In another embodiment, the mutant further comprises amino acid substitutions and/or deletions at two of positions 918, 1013, 1014, 1025 or 1028 of SEQ ID NO: 2. For example, the mutant further comprises substitutions at positions 918 and 1013; or at positions 918 and 1014; or at positions 918 and 1025; or at positions 918 and 1028; or at positions 1013 and 1014; or at positions 1013 and 1025; or at positions 1013 and 1028; or at positions 1014 and 1025; or at positions 1014 and 1028; or at positions 1025 and 1028.

In another embodiment, the mutant further comprises amino acid substitutions and/or deletions at three of positions 918, 1013, 1014, 1025 or 1028 of SEQ ID NO: 2. For example, the mutant further comprises substitutions at positions 918, 1013 and 1014; or at positions 918, 1013 and 1025; or at positions 918, 1013 and 1028; or at positions 918, 1014 and 1025; or at positions 918, 1014 and 1028; or at positions 918 and 1025 and 1028; or at positions 1013, 1014 and 1025; or at positions 1013, 1014 and 1028; or at positions 1014, 1025 and 1028.

In another embodiment, the mutant further comprises amino acid substitutions and/or deletions at four of positions 918, 1013, 1014, 1025 and 1028 of SEQ ID NO: 2. For example, the mutant further comprises substitutions at positions 918, 1013, 1014 and 1025; or at positions 918, 1013, 1025 and 1028; or at positions 1013, 1014, 1025 and 1028; or at positions 918, 1014, 1025 and 1028.

In another embodiment, the mutant further comprises amino acid substitutions and/or deletions at five of positions 918, 1013, 1014, 1025 and 1028 of SEQ ID NO: 2.

The at least one amino acid substitution, whether in the finger, REC or Lid domain or at one or more of positions 918, 1013, 1014, 1025 and 1028 of SEQ ID NO: 2, may in some embodiments be a substitution of an amino acid having a charged side chain to an amino acid having an uncharged side chain or a non-polar side chain. In some embodiments, the amino acid is substituted to an amino acid selected from the group consisting of glycine, alanine, valine, leucine, isoleucine, serine or threonine. In some embodiments, the amino acid is substituted to a glycine.

The at least two amino acid substitutions, whether in the finger, REC or Lid domain or at one or more of positions 918, 1013, 1014, 1025 and 1028 of SEQ ID NO: 2, may in some embodiments be a substitution of an amino acid having a charged side chain to an amino acid having an uncharged side chain or a non-polar side chain. In some embodiments, the amino acid is substituted to an amino acid selected from the group consisting of glycine, alanine, valine, leucine, isoleucine, serine or threonine. In some embodiments, the amino acid is substituted to a glycine.

For example, the finger domain of SEQ ID NO: 2, KGINEYINLY SQ (SEQ ID NO: 20), may be mutated to GGGAGAAGGA SG (SEQ ID NO: 22). The REC domain of SEQ ID NO: 2, LFKQILSDTE SKS (SEQ ID NO: 12) may be mutated to GGGAGASAGG SGS (SEQ ID NO: 14). The Lid domain of SEQ ID NO: 2, EDLNFGFKRG RFK (SEQ ID NO: 16) may be mutated to GGGAGGAAGG GAG (SEQ ID NO: 18).

In one embodiment of the present disclosure, the mutant Cpf1 endonuclease or orthologue thereof comprises a polypeptide sequence orthologue thereof comprising a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises at least one amino acid substitution of an amino acid having a charged side chain to an amino acid residue having an uncharged side chain in position 918, 1013, or 1014 of SEQ ID NO: 2.

In one embodiment of the present disclosure, the mutant Cpf1 endonuclease or orthologue thereof comprises a polypeptide sequence orthologue thereof comprising a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises at least two amino acid substitutions of two amino acids having a charged side chain to two amino acid residues having an uncharged side chain in two positions independently selected from the positions corresponding to residues 918, 1013, or 1014 of SEQ ID NO: 2.

In one embodiment of the present disclosure, the mutant Cpf1 endonuclease or orthologue thereof comprises a polypeptide sequence orthologue thereof comprising a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises at least one amino acid substitution of an amino acid having a charged side chain to an amino acid residue having a non-polar side chain in position 918, 1013, or 1014 of SEQ ID NO: 2.

In one embodiment of the present disclosure, the mutant Cpf1 endonuclease or orthologue thereof comprises a polypeptide sequence orthologue thereof comprising a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises at least one amino acid substitution of an amino acid having a charged side chain to an amino acid residue selected from the group consisting of glycine, alanine, valine, leucine, isoleucine, serine or threonine, in position 918, 1013, or 1014 of SEQ ID NO: 2.

In one embodiment of the present disclosure, the mutant Cpf1 endonuclease or orthologue thereof comprises a polypeptide sequence orthologue thereof comprising a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises at least one amino acid substitution of an amino acid having a charged side chain to a glycine in position 918, 1013, or 1014 of SEQ ID NO: 2.

In one embodiment of the present disclosure, the mutant Cpf1 endonuclease or orthologue thereof comprises a polypeptide sequence orthologue thereof comprising a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises at least one amino acid substitution selected from R918G, K1013G, R1014G, Q1025G or E1028G.

In one embodiment of the present disclosure, the mutant Cpf1 endonuclease or orthologue thereof comprises a polypeptide sequence orthologue thereof comprising a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises at least two amino acid substitutions selected from R918G, K1013G, R1014G, Q1025G or E1028G.

In one embodiment of the present disclosure, the mutant Cpf1 endonuclease or orthologue thereof comprises a polypeptide sequence having at least 95% sequence identity to SEQ ID NO: 2, wherein said polypeptide sequence comprises an amino acid substitution at residue K1013.

In one embodiment of the present disclosure, the mutant Cpf1 endonuclease or orthologue thereof comprises a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises an amino acid substitution at residues R918 and K1013.

In one embodiment of the present disclosure, the mutant Cpf1 endonuclease or orthologue thereof comprises a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence an amino acid substitution at residues K1013 and R1014.

In one embodiment of the present disclosure, the mutant Cpf1 endonuclease or orthologue thereof comprises a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises the amino acid substitution K1013G.

In one embodiment of the present disclosure, the mutant Cpf1 endonuclease or orthologue thereof comprises a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises the amino acid substitutions R918G and K1013G.

In one embodiment of the present disclosure, the mutant Cpf1 endonuclease or orthologue thereof comprises a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said polypeptide sequence comprises the amino acid substitutions K1013G and R1014G.

In some embodiments of the present disclosure the mutant Cpf1 endonuclease or orthologue thereof comprises a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said Cpf1 polypeptide comprises an amino acid substitution at residues K1013 and R1014 as defined in the embodiments above and is a nickase that is capable of introducing a single strand break in a target nucleic acid, such as in the first target nucleic acid, wherein the first target nucleic acid and the second target nucleic acid are identical.

In some embodiments of the present disclosure the mutant Cpf1 endonuclease or orthologue thereof comprises a polypeptide sequence having at least 95% sequence identity to SEQ ID NO: 2, such as at least 96% sequence identity to SEQ ID NO: 2, such as at least 97% sequence identity to SEQ ID NO: 2, such as at least 98% sequence identity to SEQ ID NO: 2, such as at least 99% sequence identity to SEQ ID NO: 2, such as about 100% sequence identity to SEQ ID NO: 2, wherein said Cpf1 polypeptide comprises an amino acid substitution at residues R918 and K1013 as defined in the embodiments above and is a non-specific endonuclease that is capable of introducing single strand break at non-specific sites of a nucleic acid, such as at non-specific sites of the first target nucleic acid. In some embodiments of the present disclosure the mutant Cpf1 endonuclease or orthologue thereof is a non-specific endonuclease that recognizes a target DNA sequence, but introduces a single strand break only at non-specific sites of a nucleic acid sequences. In some embodiments of the present disclosure, the mutated, non-specific Cpf1 endonuclease remains active for an extended period of time and introduces single strand breaks in several non-specific single-stranded nucleic acid sequences prior to returning into the inactive state.

In some embodiments the mutant Cpf1 endonuclease or orthologue thereof comprises a polypeptide sequence having at least 95% sequence identity to SEQ ID NO: 3 or SEQ ID NO: 4, such as at least 96% sequence identity to SEQ ID NO: 3 or SEQ ID NO: 4, for example at least 97% sequence identity to SEQ ID NO: 3 or SEQ ID NO: 4, such as at least 98% sequence identity to SEQ ID NO: 3 or SEQ ID NO: 4, for example at least 99% sequence identity to SEQ ID NO: 3 or SEQ ID NO: 4, such as 100% sequence identity to SEQ ID NO: 3 or SEQ ID NO: 4.

In some embodiments the mutant Cpf1 endonuclease or orthologue thereof comprises SEQ ID NO: 3 or SEQ ID NO: 4.

In some embodiments the mutant Cpf1 endonuclease or orthologue thereof consists of SEQ ID NO: 3 or SEQ ID NO: 4.

In one embodiment of the present disclosure, the mutant Cpf1 endonuclease or orthologue thereof comprises a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises an amino acid substitution at residue Q1025.

In another embodiment of the present disclosure, the mutant Cpf1 endonuclease or orthologue thereof comprises a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises an amino acid substitution at residue E1028.

In one embodiment of the present disclosure, the mutant Cpf1 endonuclease or orthologue thereof comprises a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises an amino acid substitution at residues Q1025 and E1028.

The substitution at position 1025 or 1028 may be as described herein. In some embodiments, position 1025 is substituted by a glycine. In some embodiments, position 1028 is substituted by a glycine. In some embodiments, positions 1025 and 1028 are both substituted by a glycine.

Cpf1 contains an RNAse active site, which is used to process its own pre-crRNA to assemble an active ribonucleoprotein to achieve interference.

The modification of the finger domain, of the REC domain and of the Lid domain of Cpf1, results in altered properties of Cpf1. The mutant proteins may thus have altered properties compared to the wild type protein, in particular the mutant Cpf1 may be able to:
Cleave a double stranded target nucleic acid;
Cleave a double stranded non-target nucleic acid;
Cleave a single stranded target nucleic acid;
Cleave a single stranded non-target nucleic acid;
Cleave single stranded DNA in a non-specific manner, optionally enhanced by double stranded target DNA;
Cleave single stranded DNA in a non-specific manner, optionally enhanced by single stranded target DNA;
Cleave only one strand of the double stranded nucleic acid target;
Cleave only the target double stranded nucleic acid; and/or
Cleave only the target single stranded nucleic acid.

The mutant Cpf1 may have one or more of the above activities. In some embodiments, the mutant Cpf1 displays improved activities compared to the wild type Cpf1.

The mutants disclosed herein may thus have different applications. For instance, mutants cleaving the target and/or non-target nucleic acid, whether single-stranded or double-stranded, in a more efficient manner than the wild-type Cpf1 from which they are derived, may be generally more useful to efficient gene editing. Mutants cleaving preferentially a double-stranded target and/or non-target nucleic acid may exhibit higher specificity than the wild-type Cpf1 from which they are derived. Mutants able to cleave single-stranded DNA non-specifically, but unable to cleave the target or non-target strand, may retain activity longer than the wild-type protein.

The mutants disclosed herein may thus have different applications. For instance, mutants cleaving the target and but not non-target single stranded nucleic acid, whether the target is single-stranded or double-stranded, in a more efficient manner than the wild type Cpf1 from which they are derived, may be generally more useful to efficient gene editing. Mutants cleaving preferentially a double-stranded target and/or non-target nucleic acid may exhibit higher specificity than the wild type Cpf1 from which they are derived. Mutants able to cleave single-stranded DNA non-specifically, but unable to cleave the target nucleic acid, may retain activity longer than the wild type protein and may be more efficient in detection of a target.

In one embodiment the Cpf1 endonuclease is a nicking endonuclease.

Polynucleotides and nucleic acid sequences encoding the mutant Cpf1 disclosed herein are also provided. The skilled person knows how to design such nucleic acid sequences encoding the desired Cpf1 mutant.

In some embodiments, the present disclosure provides a nucleic acid encoding a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to:
i) the sequence corresponding to residues 1 to 297, 310 to 323, 337 to 1005, and 1019 to 1329 of SEQ ID NO: 2, wherein said polypeptide sequence further comprises at least one amino acid substitution or deletion in the finger domain (residues 298 to 309; SEQ ID NO: 12), the REC domain (residues 324 to 336; SEQ ID NO: 16) or the Lid domain (residues 1006 to 1018; SEQ ID NO: 20) compared to SEQ ID NO: 2; and/or
ii) SEQ ID NO: 2, wherein said polypeptide sequence comprises at least one amino acid substitution in position 918, 1013, 1014, 1025 or 1028 of SEQ ID NO: 2.

In some embodiments, the present disclosure provides a nucleic acid encoding a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to:
i) the sequence corresponding to residues 1 to 323, 337 to 1005, and 1019 to 1329 of SEQ ID NO: 2, wherein said polypeptide sequence further comprises:
   a. at least two amino acid mutations in the REC domain (residues 324 to 336; SEQ ID NO: 16) compared to SEQ ID NO: 2, wherein each mutation independently is an amino acid substitution or deletion; and/or
   b. at least two amino acid substitutions in the Lid domain (residues 1006 to 1018; SEQ ID NO: 20) compared to SEQ ID NO: 2, wherein at least one of the residues at positions 917 and 1006 is a glutamic acid (E) or an aspartic acid (D);
      and/or
ii) SEQ ID NO: 2, wherein said polypeptide sequence comprises at least two amino acid substitutions in two positions independently selected from the positions corresponding to residues 918, 1013, 1014, 1025 and 1028 of SEQ ID NO: 2.

The nucleic acid sequence encoding FnCpf1 is set out in SEQ ID NO: 1.

In some embodiments, the nucleic acid sequence encodes a Cpf1 mutant wherein the finger domain has been deleted. In a particular embodiment, the nucleic acid sequence is as set out in SEQ ID NO: 41, and encodes the mutant Cpf1 of SEQ ID NO: 42.

In other embodiments, the nucleic acid sequence encodes a Cpf1 mutant wherein the REC domain has been deleted. In a particular embodiment, the nucleic acid sequence is as set out in SEQ ID NO: 33, and encodes the mutant Cpf1 of SEQ ID NO: 34.

In other embodiments, the nucleic acid sequence encodes a Cpf1 mutant wherein the amino acid residues of the REC domain have been substituted. In a particular embodiment, the nucleic acid sequence is as set out in SEQ ID NO: 35, and encodes the mutant Cpf1 of SEQ ID NO: 36.

In other embodiments, the nucleic acid sequence encodes a Cpf1 mutant wherein the lid domain has been deleted. In a particular embodiment, the nucleic acid is as set out in SEQ ID NO: 39, and encodes the mutant Cpf1 of SEQ ID NO: 40.

In other embodiments, the nucleic acid sequence encodes a Cpf1 mutant wherein the amino acid residues of the lid domain have been substituted. In a particular embodiment, the nucleic acid sequence is as set out in SEQ ID NO: 37, and encodes the mutant Cpf1 of SEQ ID NO: 38.

In some embodiments, the Cpf1 endonuclease or orthologue thereof is encoded by a nucleic acid sequence comprising or consisting of a sequence which is at least 70% identical, such as at least 75% identical, such as at least 80% identical, such as at least 85% identical, such as at least 86% identical, such as at least 87% identical, such as at least 88% identical, such as at least 89% identical, such as at least 90% identical, such as at least 91% identical, such as at least 92% identical, such as at least 93% identical, such as at least 94% identical, such as at least 95% identical, such as at least 96% identical, such as at least 97% identical, such as at least 98% identical, such as at least 99% identical to the sequence encoding the parent Cpf1, i.e. the parent sequence. In one embodiment, the parent sequence is the sequence encoding FnCpf1, as set out in SEQ ID NO: 1.

One aspect of the present disclosure relates to a vector comprising a polynucleotide or a nucleic acid sequence encoding a mutant Cpf1 endonuclease or orthologue thereof as defined above in operable combination with a promoter, wherein the Cpf1 endonuclease or orthologue thereof has at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to a polypeptide of SEQ ID NO: 2 and said polypeptide sequence comprises at least one amino acid substitution in position 918, 1013, or 1014 of SEQ ID NO: 2.

One aspect of the present disclosure relates to a vector comprising a polynucleotide or a nucleic acid sequence encoding a mutant Cpf1 endonuclease or orthologue thereof as defined above in operable combination with a promoter, wherein the Cpf1 endonuclease or orthologue thereof has at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to a polypeptide of SEQ ID NO: 2 and said polypeptide sequence comprises at least two amino acid substitution in position 918, 1013, or 1014 of SEQ ID NO: 2.

One aspect of the present disclosure relates to a vector comprising a polynucleotide or a nucleic acid sequence encoding a mutant Cpf1 endonuclease or orthologue thereof as defined above in operable combination with a promoter, wherein the Cpf1 endonuclease or orthologue thereof has at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to a polypeptide of SEQ ID NO: 2 and said polypeptide sequence comprises at least two amino acid substitution in position 1025 and 1028 of SEQ ID NO: 2.

In one embodiment, the vector further comprises a nucleic acid sequence encoding a guide RNA (crRNA) in operable combination with a promoter, wherein the crRNA binds the encoded Cpf1 endonuclease and a segment with sufficient base pairs to hybridize to a target nucleic acid. The crRNA is further described in the section below “Guide RNA (crRNA)”.

One aspect of the present disclosure relates to a system for expression of a crRNA-Cpf1 complex comprising
a. a polynucleotide as disclosed herein, or a recombinant vector as disclosed herein comprising a polynucleotide encoding a mutant Cpf1 endonuclease or orthologue thereof;
b. a polynucleotide or a recombinant vector comprising a polynucleotide encoding a guide RNA (crRNA) operably linked to a promoter.

One aspect of the present disclosure relates to a cell expressing the polynucleotide or the recombinant vector as described herein above. The Cpf1 may be expressed from a cell, in particular suitable expression hosts as are known to the skilled person. In one embodiment, Cpf1 is expressed from Escherichia coli. This can be done as is known in the art, for example by introducing a vector comprising the nucleic acid sequence encoding the desired Cpf1 effector protein or homologue as described herein above in an E. coli cell. The protein can be purified as is known in the art. In one embodiment, said polynucleotide is codon-optimized for expression in a host cell.

Without being bound by theory, the finger domain, the REC domain and the Lid domain appear to be conserved. Accordingly, the Cpf1 mutant may be a mutant of Cpf1 from Francisella novicida, Acidaminococcus sp. BV3L6, Lachnospiraceae bacterium COE1, Succiniclasticum ruminis, Candidatus Methanoplasma termitum, uncultured Clostridium sp. or Butyrivibrio fibrisolvens.

In one embodiment, the Cpf1 is FnCpf1 from Francisella novicida, and the domains are as defined herein above.

In another embodiment, the Cpf1 is derived from Acidaminococcus sp. BV3L6, as set out in SEQ ID NO: 24. The finger domain is defined by residues 275 to 285 of SEQ ID NO: 24. The REC domain is defined by residues 305 to 317 of SEQ ID NO: 24. The Lid domain is defined by residues 993 to 1005 of SEQ ID NO: 24. Position 918 of SEQ ID NO: 2 corresponds to position 909 of SEQ ID NO: 24. Position 1013 of SEQ ID NO: 2 corresponds to position 1000 of SEQ ID NO: 24. Position 1014 of SEQ ID NO: 2 corresponds to position 1001 of SEQ ID NO: 24. Position 1025 of SEQ ID NO: 2 corresponds to position 1013 of SEQ ID NO: 24. Position 1028 of SEQ ID NO: 2 corresponds to position 1016 of SEQ ID NO: 24.

In another embodiment, the Cpf1 is derived from Lachnospiraceae bacterium COE1 as set out in SEQ ID NO: 25. The finger domain is defined by residues 260 to 270 of SEQ ID NO: 25. The REC domain is defined by residues 283 to 295 of SEQ ID NO: 25. The Lid domain is defined by residues 936 to 948 of SEQ ID NO: 25. Position 918 of SEQ ID NO: 2 corresponds to position 843 of SEQ ID NO: 25. Position 1013 of SEQ ID NO: 2 corresponds to position 943 of SEQ ID NO: 25. Position 1014 of SEQ ID NO: 2 corresponds to position 944 of SEQ ID NO: 25. Position 1025 of SEQ ID NO: 2 corresponds to position 955 of SEQ ID NO: 25. Position 1028 of SEQ ID NO: 2 corresponds to position 958 of SEQ ID NO: 25.

In another embodiment, the Cpf1 is derived from Succiniclasticum ruminis, as set out in SEQ ID NO: 26. The finger domain is defined by residues 279 to 289 of SEQ ID NO: 26. The REC domain is defined by residues 309 to 321 of SEQ ID NO: 26. The Lid domain is defined by residues 997 to 1009 of SEQ ID NO: 26. Position 918 of SEQ ID NO: 2 corresponds to position 913 of SEQ ID NO: 26. Position 1013 of SEQ ID NO: 2 corresponds to position 1004 of SEQ ID NO: 26. Position 1014 of SEQ ID NO: 2 corresponds to position 1005 of SEQ ID NO: 26. Position 1025 of SEQ ID NO: 2 corresponds to position 1017 of SEQ ID NO: 26. Position 1028 of SEQ ID NO: 2 corresponds to position 1020 of SEQ ID NO: 26.

In another embodiment, the Cpf1 is derived from Candidatus Methanoplasma termitum, as set out in SEQ ID NO: 43. The finger domain is defined by residues 256 to 266 of SEQ ID NO: 43. The REC domain is defined by residues 281 to 293 of SEQ ID NO: 43. The Lid domain is defined by residues 944 to 956 of SEQ ID NO: 43. Position 918 of SEQ ID NO: 2 corresponds to position 860 of SEQ ID NO: 43. Position 1013 of SEQ ID NO: 2 corresponds to position 951 of SEQ ID NO: 43. Position 1014 of SEQ ID NO: 2 corresponds to position 952 of SEQ ID NO: 43. Position 1025 of SEQ ID NO: 2 corresponds to position 963 of SEQ ID NO: 43. Position 1028 of SEQ ID NO: 2 corresponds to position 966 of SEQ ID NO: 43.

In another embodiment, the Cpf1 is derived from uncultured Clostridium sp, as set out in SEQ ID NO: 44. The finger domain is defined by residues 266 to 270 of SEQ ID NO: 44. The REC domain is defined by residues 285 to 297 of SEQ ID NO: 44. The Lid domain is defined by residues 959 to 971 of SEQ ID NO: 44. Position 918 of SEQ ID NO: 2 corresponds to position 8776 of SEQ ID NO: 44. Position 1013 of SEQ ID NO: 2 corresponds to position 966 of SEQ ID NO: 44. Position 1014 of SEQ ID NO: 2 corresponds to position 967 of SEQ ID NO: 44. Position 1025 of SEQ ID NO: 2 corresponds to position 979 of SEQ ID NO: 44. Position 1028 of SEQ ID NO: 2 corresponds to position 982 of SEQ ID NO: 44.

In another embodiment, the Cpf1 is derived from Butyrivibrio fibrisolvens, as set out in SEQ ID NO: 27. The finger domain is defined by residues 234 to 242 of SEQ ID NO: 27. The REC domain is defined by residues 260 to 264 of SEQ ID NO: 27. The Lid domain is defined by residues 925 to 937 of SEQ ID NO: 27. Position 918 of SEQ ID NO: 2 corresponds to position 835 of SEQ ID NO: 27. Position 1013 of SEQ ID NO: 2 corresponds to position 932 of SEQ ID NO: 27. Position 1014 of SEQ ID NO: 2 corresponds to position 933 of SEQ ID NO: 27. Position 1025 of SEQ ID NO: 2 corresponds to position 944 of SEQ ID NO: 27. Position 1028 of SEQ ID NO: 2 corresponds to position 947 of SEQ ID NO: 27.

Guide RNA (crRNA)

In order to function as an endonuclease, the crRNA-Cpf1 complex requires not only the Cpf1 effector protein, but also a guide RNA (crRNA), which is responsible for recognition of the target nucleic acid to be cleaved.

The crRNA comprises or consists of a constant region and of a variable region. The constant region consists of 19-20 nucleotides and is constant for all complexes derived from a given organism. For optimal activity of the crRNA-Cpf1 complex, it may be important to design the crRNA based on the constant region specific for the organism from which Cpf1 or its homologue is derived.

The constant region may be specific for Francisella novicida and have the sequence set out in SEQ ID NO: 5.

The variable region consists of between 12 and 24 nucleotides, such as 13, 14, 15, 16, 17, 18, 19, 20, 21, 22, 23 or 24 nucleic acids. The variable region is the region of the crRNA which is thought to be responsible for target recognition. Modifying the sequence of the variable region can thus be taken advantage of in order for the crRNA-Cpf1 complex to be able to specifically cleave different target nucleic acids. In contrast to the constant region, the variable region is not organism-specific.

Accordingly, in one embodiment the crRNA consists of a constant region of 19 nucleotides and a variable region of 12 nucleotides, and the crRNA has a total length of 31 nucleotides. In another embodiment the crRNA consists of a constant region of 20 nucleotides and a variable region of 12 nucleotides, and the crRNA has a total length of 32 nucleotides. In another embodiment the crRNA consists of a constant region of 19 nucleotides and a variable region of 13 nucleotides, and the crRNA has a total length of 32 nucleotides. In another embodiment the crRNA consists of a constant region of 20 nucleotides and a variable region of 13 nucleotides, and the crRNA has a total length of 33 nucleotides. In another embodiment the crRNA consists of a constant region of 19 nucleotides and a variable region of 14 nucleotides, and the crRNA has a total length of 33 nucleotides. In another embodiment the crRNA consists of a constant region of 20 nucleotides and a variable region of 14 nucleotides, and the crRNA has a total length of 34 nucleotides. In another embodiment the crRNA consists of a constant region of 19 nucleotides and a variable region of 15 nucleotides, and the crRNA has a total length of 34 nucleotides. In another embodiment the crRNA consists of a constant region of 20 nucleotides and a variable region of 15 nucleotides, and the crRNA has a total length of 35 nucleotides. In another embodiment the crRNA consists of a constant region of 19 nucleotides and a variable region of 16 nucleotides, and the crRNA has a total length of 35 nucleotides. In another embodiment the crRNA consists of a constant region of 20 nucleotides and a variable region of 16 nucleotides, and the crRNA has a total length of 36 nucleotides. In another embodiment the crRNA consists of a constant region of 19 nucleotides and a variable region of 17 nucleotides, and the crRNA has a total length of 36 nucleotides. In another embodiment the crRNA consists of a constant region of 20 nucleotides and a variable region of 17 nucleotides, and the crRNA has a total length of 37 nucleotides. In another embodiment the crRNA consists of a constant region of 19 nucleotides and a variable region of 18 nucleotides, and the crRNA has a total length of 37 nucleotides. In another embodiment the crRNA consists of a constant region of 20 nucleotides and a variable region of 18 nucleotides, and the crRNA has a total length of 38 nucleotides. In another embodiment the crRNA consists of a constant region of 19 nucleotides and a variable region of 19 nucleotides, and the crRNA has a total length of 38 nucleotides. In another embodiment the crRNA consists of a constant region of 20 nucleotides and a variable region of 19 nucleotides, and the crRNA has a total length of 39 nucleotides. In another embodiment the crRNA consists of a constant region of 19 nucleotides and a variable region of 20 nucleotides, and the crRNA has a total length of 39 nucleotides. In another embodiment the crRNA consists of a constant region of 20 nucleotides and a variable region of 20 nucleotides, and the crRNA has a total length of 40 nucleotides. In another embodiment the crRNA consists of a constant region of 19 nucleotides and a variable region of 21 nucleotides, and the crRNA has a total length of 40 nucleotides. In another embodiment the crRNA consists of a constant region of 20 nucleotides and a variable region of 21 nucleotides, and the crRNA has a total length of 41 nucleotides. In another embodiment the crRNA consists of a constant region of 19 nucleotides and a variable region of 22 nucleotides, and the crRNA has a total length of 41 nucleotides. In another embodiment the crRNA consists of a constant region of 20 nucleotides and a variable region of 22 nucleotides, and the crRNA has a total length of 42 nucleotides. In another embodiment the crRNA consists of a constant region of 19 nucleotides and a variable region of 23 nucleotides, and the crRNA has a total length of 42 nucleotides. In another embodiment the crRNA consists of a constant region of 20 nucleotides and a variable region of 23 nucleotides, and the crRNA has a total length of 43 nucleotides. In another embodiment the crRNA consists of a constant region of 19 nucleotides and a variable region of 24 nucleotides, and the crRNA has a total length of 43 nucleotides. In another embodiment the crRNA consists of a constant region of 20 nucleotides and a variable region of 24 nucleotides, and the crRNA has a total length of 44 nucleotides.

The skilled person will have no difficulty in designing a variable region capable of binding the desired target nucleic acid. The variable region has a sequence which is the reverse complement of the target nucleic acid.

The crRNA thus consists of a constant region of 19 or 20 nucleotides, and of a variable region consisting of between 12 and 24 nucleotides, such that said crRNA is 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, 41 nucleotides in length, 42 nucleotides in length, 43 nucleotides in length, 44 nucleotides in length, with the proviso that the 5′-terminal nucleotides consist of a PAM as described in the sections “First target nucleic acid” and “Second target nucleic acid”.

Once a guide RNA sequence has been designed, the guide RNA can be synthesised by known methods. For example, DNA oligonucleotides corresponding to the reverse complemented sequence of the target site may be ordered from a company selling oligonucleotides. These oligonucleotides may contain a 24 base long T7 priming sequence. These DNA duplexes may then be used as template in a transcription reaction carried with T7 RNA polymerase. For example, the reaction may consist of incubation at 37° C. for at least 1 hour. The reaction may be stopped using 2× stop solution, for example 50 mM EDTA, 20 mM Tris-HCl pH 8.0 and 8 M Urea. The RNA may be purified by methods known in the art such as LiCl precipitation.

crRNA-Cpf1 Complex

When associated with the appropriate crRNA, Cpf1 forms a crRNA-Cpf1 complex. The crRNA-Cpf1 complexes formed by some Cpf1 mutants of the present disclosure is capable of
introducing single strand breaks in a first target nucleic acid, and
specifically recognizing a second target nucleic acid.

This is in contrast to a crRNA-Cpf1 complex formed with a wild type Cpf1 endonuclease, and which is capable of recognizing a specific nucleic acid and cleaving both strands of said specific nucleic acid in a staggered manner.

The crRNA may be modified in order for the crRNA-Cpf1 complex to be able to recognise different target nucleic acids. In particular, the variable region of the crRNA can be designed in order to recognise different targets. The sequence of the variable region of the crRNA needs to be complementary to the target nucleic acid.

It will be understood that a Cpf1 or homologue thereof derived from a given organism is preferably to be used in combination with a crRNA having a constant region as naturally found in the same organism. Thus, FnCpf1 or a homologue thereof is preferably assembled with a crRNA having a constant region as set out in SEQ ID NO: 5.

In order for the crRNA-Cpf1 complex to function efficiently as an endonuclease or restriction enzyme, the ratio of Cpf1 to crRNA is preferably adjusted. Preferably, the molar ratio of Cpf1 to crRNA is between 0.5:3.0 and 1.0:1.0, such as 0.7:2.5, such as 0.8:2.0, such as 0.9:1.75, such as 0.95:1.5, such as 1.0:1.4, such as 1.0:1.3, such as 1.1:1.2.

In some embodiments, the Cpf1 mutant which is capable of forming a crRNA-Cpf1 complex having the ability to introduce single strand breaks in a first target nucleic acid, and specifically recognize and optionally cleave a second target nucleic acid as described herein is a Cpf1 mutant having comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to:
i) the sequence corresponding to residues 1 to 297, 310 to 323, 337 to 1005, and 1019 to 1329 of SEQ ID NO: 2, wherein said polypeptide sequence further comprises at least one amino acid substitution or deletion in the finger domain (residues 298 to 309; SEQ ID NO: 12), the REC domain (residues 324 to 336; SEQ ID NO: 16) or the Lid domain (residues 1006 to 1018; SEQ ID NO: 20) compared to SEQ ID NO: 2; and/or
ii) SEQ ID NO: 2, wherein the polypeptide sequence comprises at least one amino acid substitution in position 918, 1013, 1014, 1025 or 1028 of SEQ ID NO: 2.

In some embodiments, the cleavage of the second target nucleic acid is single-stranded cleavage. In other embodiments, the cleavage of the second target nucleic acid is double-stranded cleavage. Preferably, cleavage of the second target nucleic acid is single-stranded cleavage. The cleavage may be further enhanced by the presence of an activator DNA, which may be single-stranded or double-stranded.

As can be seen in the examples, a mutant Cpf1 having a Q1025E substitution is capable of such non-specific DNA cleavage of a single stranded DNA. The cleavage is enhanced by the presence of single stranded target DNA or double stranded target DNA. Likewise, a mutant Cpf1 deleted for the REC domain as set out in SEQ ID NO: 34 and a mutant Cpf1 wherein the REC domain has been entirely substituted (SEQ ID NO: 36) are also capable of such non-specific DNA cleavage of a single stranded DNA. The cleavage is enhanced by the presence of single stranded target DNA or double stranded target DNA. A Cpf1 mutant in which the Lid domain has been entirely substituted is capable of such non-specific DNA cleavage of a single stranded DNA, albeit to a lesser extent than the above mutants; however in this case cleavage appears to be enhanced by the presence of single stranded target DNA only. A K1013G, R1014G mutant is also capable of such non-specific cleavage.

Herein are thus provided Cpf1 mutants which, when in complex with a crRNA, cleave a nucleic acid sequence more efficiently than the wild type Cpf1 endonuclease they are derived from. In some embodiments, the mutant Cpf1 endonuclease cuts both the target strand and the non-target strand more efficiently than the wild type Cpf1 endonuclease. In other embodiments, the mutant Cpf1 endonuclease alternatively or additionally cuts only one of the target strand and non-target strand more efficiently than the wild type Cpf1 endonuclease. In other embodiments, the mutant Cpf1 endonuclease alternatively or additionally is capable of introducing single-stranded breaks in a non-specific manner, as detailed herein below.

For example, a Cpf1 mutant comprising a mutation or a substitution at position 1025 of SEQ ID NO: 2 may cleave a nucleic acid sequence more efficiently than the wild type Cpf1 endonuclease of SEQ ID NO: 2.

A Cpf1 mutant comprising a mutation or a substitution at positions 1025 and 1028 of SEQ ID NO: 2 may cleave a nucleic acid sequence more efficiently than the wild type Cpf1 endonuclease of SEQ ID NO: 2.

A Cpf1 mutant comprising at least one mutation or substitution in the finger domain defined by residues 298 to 309 of SEQ ID NO: 2 may cleave a nucleic acid sequence more efficiently than the wild type Cpf1 endonuclease of SEQ ID NO: 2.

A Cpf1 mutant comprising at least one mutation or substitution in the REC domain defined by residues 324 to 336 of SEQ ID NO: 2 may cleave a nucleic acid sequence more efficiently than the wild type Cpf1 endonuclease of SEQ ID NO: 2.

A Cpf1 mutant comprising a mutation or a substitution in the Lid domain defined by residues 1006 to 1018 of SEQ ID NO: 2 may cleave a nucleic acid sequence more efficiently than the wild type Cpf1 endonuclease of SEQ ID NO: 2.

### First Target Nucleic Acid

The mutant Cpf1 endonuclease of the present disclosure, after forming a crRNA-Cpf1 complex, is capable to introduce a single strand break in the first target nucleic acid. The first target nucleic acid is a nucleic acid sequence which may not be specifically recognized by the crRNA-Cpf1 complex of the present disclosure, but nevertheless cleaved.

In some embodiments, the first target nucleic acid is identical to the second target nucleic acid and it is recognized and cleaved by the crRNA-Cpf1 complex. Hence, the first target nucleic acid may be as defined in the section below “Second target nucleic acid”.

In some embodiments, the first target nucleic acid is different from the second target nucleic acid and it is cleaved by the crRNA-Cpf1 complex only after hybridization of the crRNA-Cpf1 complex to the second target nucleic acid. Hence, in some embodiments the first target nucleic acid does not comprise a protospacer adjacent motif (PAM).

### Second Target Nucleic Acid

Recognition and binding of the crRNA-Cpf1 complex to a second target nucleic acid relies on the crRNA binding to the second target nucleic acid. This is dependent on the presence of a PAM (protospacer adjacent motif) in the target nucleic acid. In some embodiments, Cpf1 is FnCpf1 and the PAM sequence is 5′-YRN-3′ or 5′-RYN-3′, where Y is a pyrimidine such as A or G, R is a purine such as T, U or C, and N is any nucleotide. Preferably, R is T or C.

In one embodiment, the PAM consists of the sequence 5′-TTN-3′. In another embodiment, the PAM consists of the sequence 5′-CCN-3′. In another embodiment, the PAM consists of the sequence 5′-TAN-3′. In another embodiment, the PAM consists of the sequence 5′-TCN-3′. In another embodiment, the PAM consists of the sequence 5′-TGN-3′. In another embodiment, the PAM consists of the sequence 5′-CTN-3′. In another embodiment, the PAM consists of the sequence 5′-CAN-3′. In another embodiment, the PAM consists of the sequence 5′-CGN-3′. In another embodiment, the PAM consists of the sequence 5′-ATN-3′. In another embodiment, the PAM consists of the sequence 5′-AAN-3′. In another embodiment, the PAM consists of the sequence 5′-ACN-3′. In another embodiment, the PAM consists of the sequence 5′-AGN-3′. In another embodiment, the PAM consists of the sequence 5′-TTN-3′. In another embodiment, the PAM consists of the sequence 5′-TAN-3′. In another embodiment, the PAM consists of the sequence 5′-TCN-3′. In another embodiment, the PAM consists of the sequence 5′-TGN-3′.

The crRNA preferably does not hybridize to the PAM itself. The choice of the PAM sequence may be dictated by the organism from which the Cpf1 to be used to cleave the target nucleic acid is derived.

The second target nucleic acid may comprise or consist of a recognition sequence comprising a sequence of at least 15 consecutive nucleotides, such as at least 16 consecutive nucleotides, such as at least 17 consecutive nucleotides, such as at least 18 consecutive nucleotides, such as at least 19 consecutive nucleotides, such as at least 20 consecutive nucleotides, such as at least 21 consecutive nucleotides, such as at least 22 consecutive nucleotides, such as at least 23 consecutive nucleotides, such as at least 24 consecutive nucleotides, such as at least 25 consecutive nucleotides, such as at least 26 consecutive nucleotides, such as at least 27 consecutive nucleotides, with the proviso that the 3 nucleic acids at the 5′-end consist of a PAM sequence.

The second target nucleic acid sequence is DNA or RNA. The second target nucleic acid sequence may be DNA selected from the group consisting of genomic DNA, chromatin, nucleosomes, plasmid DNA, methylated DNA, synthetic DNA, and DNA fragments, for example PCR products.

In some embodiments, the second target nucleic acid is RNA.

In some embodiments, the second target nucleic acid is preferably DNA.

The second target nucleic acid may be any nucleic acid which it may be desirable to cleave specifically. The second target nucleic acid may be purified prior to contacting it with the crRNA-Cpf1 complex, by methods known in the art.

In some embodiments, the second target nucleic acid may be in recognized and hybridized in vivo.

Use of a crRNA-Cpf1 endonuclease complex for introducing a single strand break One aspect of the present disclosure relates to use of a crRNA-Cpf1 complex for introducing a single strand break in a first target nucleic acid, wherein:
a. a Cpf1 endonuclease or orthologue thereof is contacted with a guide RNA (crRNA), thereby obtaining a crRNA-Cpf1 complex capable of recognising a second target nucleic acid, the second target nucleic acid comprising a protospacer adjacent motif (PAM), and
b. the crRNA-Cpf1 complex is contacted with the first target nucleic acid;
whereby a single strand break is made in the first target sequence.

The crRNA-Cpf1 complex comprises a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to:
i) the sequence corresponding to residues 1 to 297, 310 to 323, 337 to 1005, and 1019 to 1329 of SEQ ID NO: 2, wherein said polypeptide sequence further comprises at least one amino acid substitution or deletion in the finger domain (residues 298 to 309; SEQ ID NO: 12), the REC domain (residues 324 to 336; SEQ ID NO: 16) or the Lid domain (residues 1006 to 1018; SEQ ID NO: 20) compared to SEQ ID NO: 2; and/or
ii) SEQ ID NO: 2, wherein said polypeptide sequence comprises at least one amino acid substitution in position 918, 1013, 1014, 1025 or 1028 of SEQ ID NO: 2.

The crRNA-Cpf1 complex comprises a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to:
i) the sequence corresponding to residues 1 to 323, 337 to 1005, and 1019 to 1329 of SEQ ID NO: 2, wherein said polypeptide sequence further comprises:
   a. at least one amino acid mutation in the REC domain (residues 324 to 336; SEQ ID NO: 16) compared to SEQ ID NO: 2, wherein each mutation independently is an amino acid substitution or deletion; and/or
   b. at least two amino acid substitutions the Lid domain (residues 1006 to 1018; SEQ ID NO: 20) compared to SEQ ID NO: 2, wherein at least one of the residues at positions 917 and 1006 is a glutamic acid (E) or an aspartic acid (D);
      and/or
ii) SEQ ID NO: 2, wherein said polypeptide sequence comprises at least one amino acid substitution in position 918, 1013, 1014, 1025 or 1028 of SEQ ID NO: 2.

The crRNA-Cpf1 complex in other embodiments comprises a mutant Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least one amino acid substitution in position 918, 1013, or 1014 of SEQ ID NO: 2, said mutant Cpf1 is described in detail herein above in the section “Mutant Cpf1”.

In one embodiment the crRNA hybridises to the second target nucleic acid and the Cpf1 introduces a single strand break in a specific recognition nucleotide sequence of the first target nucleic acid.

In one embodiment the crRNA hybridises to the second target nucleic acid and the Cpf1 introduces a single strand break in a specific recognition nucleotide sequence of the first target nucleic acid, wherein the first and the second target nucleic acid are identical or overlapping. For example, the first and the second target nucleic acid may both comprise the same recognition sequence and the same PAM.

In one embodiment the crRNA hybridises to the second target nucleic acid and the Cpf1 introduces a single strand break in a specific recognition nucleotide sequence of the first target nucleic acid, wherein the first and the second target nucleic acid are the same nucleic acid.

The characteristics of the crRNA are described herein above in the section “Guide RNA (crRNA)”.

Thus, the first and the second target nucleic may be identical or different. In one embodiment the first and the second target nucleic are identical. For example, the first and the second target nucleic are the same target nucleic acid.

In one embodiment the crRNA hybridises to the second target nucleic acid and the Cpf1 introduces a single strand break into one or more nonspecific nucleotide sequences of the first target nucleic acid.

In one embodiment, the first and the second target nucleic are different and the first target nucleic acid does not comprise a PAM sequence and thus is not recognized by the crRNA-Cpf1 complex, whereas the second target nucleic acid comprises a PAM sequence and is recognized by the crRNA-Cpf1 complex.

The mutant Cpf1 comprised in the crRNA-Cpf1 complex useful for introducing a single-stranded break in a nucleic acid sequence may be any of the mutants described herein. However, some mutants may be particularly advantageous. For example, a mutant Cpf1 having a Q1025G substitution, a mutant Cpf1 deleted for the REC domain as set out in SEQ ID NO: 34, a mutant Cpf1 wherein the REC domain has been entirely substituted (SEQ ID NO: 36), a mutant Cpf1 mutant in which the Lid domain has been entirely substituted are all capable of such non-specific DNA cleavage of a single stranded DNA, and are particularly useful for cleaving a second target nucleic acid which is different from the first target nucleic acid. Mutant Cpf1 having mutations at positions 1013 and 1014, or at positions 918 and 1013, may also be of interest.

In one embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises an amino acid substitution at position 1025 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a deletion of one or more amino acid residues of the REC domain defined by residues 298 to 309 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a deletion of one or more amino acid residues of the Lid domain defined by residues 324 to 336 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a mutation of two or more amino acid residues of the REC domain defined by residues 324 to 336 of SEQ ID NO: 2, wherein each mutation independently is an amino acid substitution or deletion.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a deletion of one or more amino acid residues of the Lid domain defined by residues 1006 to 1018 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a substitution of two or more amino acid residues of the Lid domain defined by residues 1006 to 1018 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises an amino acid substitution at one or more of positions 918, 1013, 1014, 1025 and 1028 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises amino acid substitutions at position 1013 and 1014 of SEQ ID NO: 2.

The substitution may in some embodiments be a substitution of an amino acid having a charged side chain to an amino acid having an uncharged side chain or a non-polar side chain. In some embodiments, the amino acid is substituted to an amino acid selected from the group consisting of glycine, alanine, valine, leucine, isoleucine, serine or threonine. In some embodiments, the amino acid is substituted to a glycine.

The deletion may in some embodiments be a deletion of one, more than one, or all the amino acid residues of the REC domain, the Lid domain or the finger domain. In some embodiments, some amino acids are substituted, while others are deleted. In some embodiments, the Cpf1 mutant comprises amino acid deletions and/or substitutions in more than one domain, e.g. in the REC domain and the Lid domain, in the REC domain and the finger domain, in the Lid domain and the finger domain, or in the REC domain, the Lid domain and the finger domain. The Cpf1 mutant may further comprise amino acid substitutions or deletions at one or more of positions 918, 1013, 1014, 1025 or 1028.

For example the crRNA-Cpf1 complex is used for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises amino acid substitutions at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises substitutions of an amino acid having a charged side chain to an amino acid having an uncharged side chain at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises substitutions of an amino acid having a charged side chain to an amino acid having an uncharged side chain at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises substitutions of an amino acid having a charged side chain to an amino acid having a non-polar side chain at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises substitutions of an amino acid having a charged side chain to an amino acid having a non-polar side chain at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises substitutions of an amino acid having a charged side chain to chain to a glycine, alanine, valine, leucine, isoleucine, serine or threonine in at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises substitutions of an amino acid having a charged side chain to chain to a glycine, alanine, valine, leucine, isoleucine, serine or threonine at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises substitutions of an amino acid having a charged side chain to chain to a glycine at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises substitutions of an amino acid having a charged side chain to chain to a glycine at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises substitutions of an amino acid having a charged side chain to chain to a glycine at position 1013 and 1014 of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises the substitutions K1013G and R1014G of SEQ ID NO: 2.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises or consists of a polypeptide having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to SEQ ID NO: 3.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises or consists of a polypeptide having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to SEQ ID NO: 3.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises or consists of SEQ ID NO: 3.

In one embodiment the present disclosure relates to use of a crRNA-Cpf1 complex for introducing a single strand break in a specific recognition nucleotide sequence of the first target nucleic acid, wherein the first target nucleic acid and the second target nucleic acid are the same nucleic acid, and wherein the Cpf1 endonuclease or orthologue thereof comprises or consists of SEQ ID NO: 3.

Some of the mutant Cpf1 endonucleases described herein are capable of introducing single strand breaks in a target nucleic acid, referred herein as the first target nucleic acid, which differs from the nucleic acid sequence recognized and hybridized by the crRNA, referred herein as the second target nucleic acid.

In some embodiments, the present disclosure relates to the use of a crRNA-Cpf1 complex for introducing a single strand break at one or more nonspecific nucleotide sequences of the first target nucleic acid, wherein the first target nucleic acid and the second target nucleic acid are different, and wherein the Cpf1 endonuclease or orthologue thereof has a mutation at position 1025 of SEQ ID NO: 2. In a particular embodiment, the mutation is a Q1025G mutation.

In other embodiments, the present disclosure relates to the use of a crRNA-Cpf1 complex for introducing a single strand break at one or more nonspecific nucleotide sequences of the first target nucleic acid, wherein the first target nucleic acid and the second target nucleic acid are different, and wherein the Cpf1 endonuclease or orthologue thereof has at least one amino acid substitution or deletion of the REC domain corresponding to residues 324 to 336 of SEQ ID NO: 2. For example, the mutant Cpf1 has a complete deletion of the REC domain, and has the sequence as set out in SEQ ID NO: 34. In a specific embodiment, the entire REC domain has been substituted and the mutant Cpf1 has for example the sequence set out in SEQ ID NO: 36.

In other embodiments, the present disclosure relates to the use of a crRNA-Cpf1 complex for introducing a single strand break at one or more nonspecific nucleotide sequences of the first target nucleic acid, wherein the first target nucleic acid and the second target nucleic acid are different, and wherein the Cpf1 endonuclease or orthologue thereof has at least one amino acid substitution or deletion of the Lid domain corresponding to residues 324 to 336 of SEQ ID NO: 2. For example, the entire Lid domain has been substituted and the mutant Cpf1 has the sequence set out in SEQ ID NO: 42.

In other embodiments, the present disclosure relates to the use of a crRNA-Cpf1 complex for introducing a single strand break at one or more nonspecific nucleotide sequences of the first target nucleic acid, wherein the first target nucleic acid and the second target nucleic acid are different, and wherein the Cpf1 endonuclease or orthologue thereof has at least one amino acid substitution or deletion of the Lid domain corresponding to residues 1006 to 1018 of SEQ ID NO: 2. For example, the entire Lid domain has been substituted and the mutant Cpf1 has the sequence set out in SEQ ID NO: 38.

In some embodiments, the crRNA-Cpf1 complex comprises amino acid substitutions or deletions in more than one of the REC, Lid or finger domain, for example in the REC domain and in the Lid domain; in the REC domain and in the finger domain; in the Lid domain and in the finger domain; or in the REC domain, the finger domain and the Lid domain. The mutant Cpf1 may further comprise amino acid substitutions or deletions at one or more of positions 918, 1013, 1014, 1025 or 1028 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are different, and wherein the Cpf1 endonuclease or orthologue thereof comprises substitutions of an amino acid having a charged side chain to an amino acid having an uncharged side chain at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are the same nucleic acid, and wherein the Cpf1 endonuclease or orthologue thereof comprises substitutions of an amino acid having a charged side chain to an amino acid having an uncharged side chain at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are identical, and wherein the Cpf1 endonuclease or orthologue thereof comprises substitutions of an amino acid having a charged side chain to an amino acid having a non-polar side chain at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are the same nucleic acid, and wherein the Cpf1 endonuclease or orthologue thereof comprises substitutions of an amino acid having a charged side chain to an amino acid having a non-polar side chain at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are identical, and wherein the Cpf1 endonuclease or orthologue thereof comprises substitutions of an amino acid having a charged side chain to chain to a glycine, alanine, valine, leucine, isoleucine, serine or threonine in at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are the same nucleic acid, and wherein the Cpf1 endonuclease or orthologue thereof comprises substitutions of an amino acid having a charged side chain to chain to a glycine, alanine, valine, leucine, isoleucine, serine or threonine at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are identical, and wherein the Cpf1 endonuclease or orthologue thereof comprises substitutions of an amino acid having a charged side chain to chain to a glycine at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are the same nucleic acid, and wherein the Cpf1 endonuclease or orthologue thereof comprises substitutions of an amino acid having a charged side chain to chain to a glycine at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are identical, and wherein the Cpf1 endonuclease or orthologue thereof comprises substitutions of an amino acid having a charged side chain to chain to a glycine at position 918 and 1013 of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are the same nucleic acid, and wherein the Cpf1 endonuclease or orthologue thereof comprises the substitutions R918G and K1013G of SEQ ID NO: 2.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are identical, and wherein the Cpf1 endonuclease or orthologue thereof comprises or consists of a polypeptide having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as about 100% sequence identity to SEQ ID NO: 4.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are the same nucleic acid, and wherein the Cpf1 endonuclease or orthologue thereof comprises or consists of a polypeptide having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as about 100% sequence identity to SEQ ID NO: 4.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are identical, and wherein the Cpf1 endonuclease or orthologue thereof comprises or consists of SEQ ID NO: 4.

In one embodiment the present disclosure relates to use of a crRNA-Cpf1 complex for introducing a single strand break at one or more nonspecific nucleotide sequences of the first target nucleic acid, wherein the first target nucleic acid and the second target nucleic acid are the same nucleic acid, and wherein the Cpf1 endonuclease or orthologue thereof comprises or consists of SEQ ID NO: 4.

The mutants may further comprise additional mutations or deletions as described herein above in the section “Mutant Cpf1”.

The crRNA-Cpf1 complex may be used for introducing single strand breaks in a first target nucleic acid in an ex-vivo method. The crRNA-Cpf1 complex may also be used for introducing single strand breaks in a first target nucleic acid in an in-vivo method.

In some embodiments, the efficiency of the introduction of single-strand breaks as described above is enhanced by the presence of activator DNA. The activator DNA may be single-stranded or double-stranded. In some embodiments, the activator DNA is the target DNA, corresponding to the second target nucleic acid.

Use of a crRNA-Cpf1 Endonuclease Complex for Genome Editing

Some of the mutant Cpf1 endonucleases of the present disclosure are capable of introducing single strand breaks only in the target sequence, which is hybridized by the crRNA of the crRNA-Cpf1 complex. Thus, in some embodiments when the crRNA of a crRNA-Cpf1 complex recognizes and hybridizes to a second target sequence, the DNAse activity of the Cpf1 of said complex will be activated and it will introduce single strand breaks specifically at specific sites of the second target nucleic acid sequence. Moreover, unspecific nucleic acid sequences will not be cleaved by the mutant Cpf1. This constitutes a great advantage over wild type Cpf1, which instead has non-specific ssDNA activity. In fact, single strand non-specific DNA cleavage is a dangerous activity for the cells since it targets the DNA that opens during normal cellular activity such as replication and transcription. The major outcome of this non-specific ssDNA activity is the production of random breaks that can results in unwanted mutagenesis and even cellular death.

The mutant Cpf1 endonucleases of the present disclosure may advantageously be used for genome editing in a safer manner compared to wild type Cpf1.

Hence, one aspect of the present disclosure relates to an in vitro method of introducing a site-specific, double-stranded break at a second target DNA sequence in a mammalian cell, the method comprising introducing into the mammalian cell a crRNA-Cpf1 complex, wherein the Cpf1 is a mutant Cpf1 endonuclease or orthologue thereof as described herein, and wherein the crRNA is specific for the second target nucleic acid.

In some embodiments, the mammalian cell is not a reproductive cell or an embryonic cell.

In some embodiments, the mutant Cpf1 endonuclease disclosed herein and used in an in vitro method of introducing a site-specific, double-stranded break at a second target DNA sequence in a mammalian cell, may be a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions in position 1025 and 1028 of SEQ ID NO: 2.

In some embodiments, the mutant Cpf1 endonuclease disclosed herein and used in an in vitro method of introducing a site-specific, double-stranded break at a second target DNA sequence in a mammalian cell, may be a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to an amino acid having an uncharged side chain in position 1025 and 1028 of SEQ ID NO: 2.

In some embodiments, the mutant Cpf1 endonuclease disclosed herein and used in an in vitro method of introducing a site-specific, double-stranded break at a second target DNA sequence in a mammalian cell, may be a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to an amino acid having a non-polar side chain in position 1025 and 1028 of SEQ ID NO: 2.

In some embodiments, the mutant Cpf1 endonuclease disclosed herein and used in an in vitro method of introducing a site-specific, double-stranded break at a second target DNA sequence in a mammalian cell, may be a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to a glycine, alanine, valine, leucine, isoleucine, serine or threonine in position 1025 and 1028 of SEQ ID NO: 2.

In some embodiments, the mutant Cpf1 endonuclease disclosed herein and used in an in vitro method of introducing a site-specific, double-stranded break at a second target DNA sequence in a mammalian cell, may be a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to a glycine in position 1025 and 1028 of SEQ ID NO: 2.

For example mutant Cpf1 endonuclease used in the in vitro method of introducing a site-specific, double-stranded break at a second target DNA sequence in a mammalian cell, is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises the amino acid substitutions Q1025G and E1028G.

For example mutant Cpf1 endonuclease used in the in vitro method of introducing a site-specific, double-stranded break at a second target DNA sequence in a mammalian cell, is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 32.

The mutants may further comprise additional mutations or deletions as described herein above in the section “Mutant Cpf1”.

For example, mutant Cpf1 endonuclease used in the in vitro method of introducing a site-specific, double-stranded break at a second target DNA sequence in a mammalian cell, may be a Cpf1 endonuclease or orthologue thereof comprising at least two amino acid substitutions in the Lid domain as defined herein and one or more amino acid substitutions in any one of positions 918, 1013, 1014, 1025 or 1028 of SEQ ID NO: 2.

For example, mutant Cpf1 endonuclease used in the in vitro method of introducing a site-specific, double-stranded break at a second target DNA sequence in a mammalian cell, may be a Cpf1 endonuclease or orthologue thereof comprising at least two amino acid mutations in the REC domain as defined herein, wherein each mutation independently is an amino acid substitution or deletion, and one or more amino acid substitutions in any one of positions 918, 1013, 1014, 1025 or 1028 of SEQ ID NO: 2.

For example, mutant Cpf1 endonuclease used in the in vitro method of introducing a site-specific, double-stranded break at a second target DNA sequence in a mammalian cell, may be a Cpf1 endonuclease or orthologue thereof comprising at least two amino acid substitutions in the REC domain as defined herein and one or more amino acid substitutions in any one of positions 918, 1013, 1014, 1025 or 1028 of SEQ ID NO: 2.

For example, mutant Cpf1 endonuclease used in the in vitro method of introducing a site-specific, double-stranded break at a second target DNA sequence in a mammalian cell, may be a Cpf1 endonuclease or orthologue thereof comprising at least two amino acid deletions in the REC domain as defined herein and one or more amino acid substitutions in any one of positions 918, 1013, 1014, 1025 or 1028 of SEQ ID NO: 2.

Use of a crRNA-Cpf1 Endonuclease Complex for Detection and/or Quantification of a Target DNA Sequence

Some of the mutant Cpf1 endonucleases of the present disclosure are capable of introducing single strand breaks only in a first target sequence, which is not hybridized by the crRNA of the crRNA-Cpf1 complex. Thus, in some embodiments when the crRNA of a crRNA-Cpf1 complex recognizes and hybridizes to a second target sequence, the DNAse activity of the Cpf1 of said complex will be activated and it will introduce single strand breaks unspecifically at random sites of a nucleic acid sequence, for example at random sites of the first target sequence. Moreover, the second target nucleic acid will not be cleaved by the Cpf1, which will therefore stay in an active state for a longer period of time and cleave more than one first target sequences. Provided that the first target sequence is labelled in a way that a signal will be released upon cleavage of said first target sequence, the described method will thus allow detection of the second target sequence.

These mutant Cpf1 endonucleases, when in a crRNA-Cpf1 complex, can be used to detect and quantify a second target sequence, with the help of a provided labelled first target sequence.

Hence, one aspect of the present disclosure relates to a method for detection of a second target nucleic acid in a sample, the method comprising:
a. Providing a crRNA-Cpf1 complex, wherein the Cpf1 is a Cpf1 endonuclease or orthologue thereof as described herein, and wherein the crRNA is specific for the second target nucleic acid;
b. Providing a labelled ssDNA, wherein the ssDNA is labelled with at least one set of interactive labels comprising at least one dye and at least one quencher;
c. Contacting the crRNA-Cpf1 complex and the ssDNA with the sample, wherein the sample comprises at least one second target nucleic acid; and
d. Detecting cleavage of the ssDNA, by detecting a signal from the dye
   thereby detecting the presence of the second target nucleic acid in the sample.

In step c. the crRNA-Cpf1 complex and the ssDNA are contacted with at least one second target nucleic acid, and the recognition and binding of the crRNA with the second target nucleic acid results in activation of the crRNA-Cpf1 complex, which is then capable of introducing single strand breaks, such as cleaving, the ssDNA.

Hence, step c. may comprise activation of the crRNA-Cpf1 complex.

The method may further comprise the step of determining the level and/or concentration of the second target nucleic acid, wherein the level and/or concentration of the second target nucleic acid is correlated to the cleaved ssDNA.

As explained above, in some embodiments the mutant Cpf1 endonuclease disclosed herein will not cleave the second target nucleic acid and thus will stay active for a period of time sufficient for cleaving two or more first target nucleic acids, which in the method described herein may be the ssDNA or a fragment thereof. The more first target nucleic acid molecules are cleaved by the ccRNA-Cpf1 complex after hybridization of the ccRNA-Cpf1 complex to a second target nucleic acid, the higher the signal and thus the higher the sensitivity of the method. This is an advantage of the disclosed mutant Cpf1 over other Cpf1 endonucleases.

Hence, the method disclosed herein has high sensitivity and may allow detection of the second target nucleic acid at concentrations in the nanomolar range and below, such as at concentrations in the picomolar range and below, such as at concentrations in the femtomolar range or below. For example, the method disclosed herein allows detection of a second target nucleic acid at concentrations in the attomolar range.

In some embodiments, the mutant Cpf1 endonuclease disclosed herein will cleave the second target nucleic acid and thus will stay active only until the cleaved second target nucleic acid is released.

The ssDNA may be labelled in at least one base in any position along the chain. For example, the ssDNA is labelled in one base in any position along the chain, such as in at least two bases in any position along the chain, such as in at least three bases in any position along the chain, such as in at least four bases in any position along the chain.

The ssDNA may be labelled with at least one set of interactive labels comprising at least one dye and at least one quencher.

In one embodiment, the at least one dye is a fluorophore.

Thus, the cleavage of the ssDNA in step d. of the method comprises detecting a fluorescent signal resulting from cleavage of the ssDNA.

In one embodiment the at least one fluorophore is selected from the group comprising black hole quencher (BHQ) 1, BHQ2, and BHQ3, Cosmic Quencher (e.g. from Biosearch Technologies, Novato, USA), Excellent Bioneer Quencher (EBQ) (e.g. from Bioneer, Daejeon, Korea) or a combination hereof.

In one embodiment the at least one quencher is selected from the group comprising black hole quencher (BHQ) 1, BHQ2, and BHQ3 (from Biosearch Technologies, Novato, USA).

A fluorophore which may be useful in the present invention may include any fluorescent molecule known in the art. Examples of fluorophores are: Cy2™ Cfflfi), YO-PRn™-1 (509), YDYO™-1 (509), Calrein (517), FITC (518), FluorX™ (519), Alexa™ (520), Rhodamine 110 (520), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-I (533), TOTOI (533), JOE (548), 30 BODIPY530/550 (550), Dil (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 35 (615), Texas Red (615), Nile Red (628), YO-PRO™-3 (631), YOYO™-3 (631), RP3649PC00 phycocyanin (642), C-Phycocyanin (648), TO-PRO™-3 (660), TOT03 (660), DID DiIC (5) (665), Cy5™ (670), Thiadicarbocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), 6-Carboxyfluorescein (6-FAM), Fluorescein (520), Fluorescein-C3 (520), Pulsar 650 (566), Quasar 570 (667), Quasar 670 (705) and Quasar 705 (610). The number in parenthesis is a maximum emission wavelength in nanometers.

It is noteworthy that a non-fluorescent black quencher molecule capable of quenching a fluorescence of a wide range of wavelengths or a specific wavelength may be used in the present invention.

Suitable pairs of fluorophores/quenchers are known in the art.

The method may be used to detect presence and levels of any nucleic acid and thus the sample may be any sample comprising nucleic acid and appropriately treated, for example to eliminate proteases. The sample may comprise DNA and/or RNA. The sample may be a sample suspected of comprising the second target nucleic acid. The sample may be culture extract of any prokaryotic or eukaryotic cell culture, body fluid of a mammal, such as of a human.

The second target nucleic acid may be a nucleic acid fragment of a viral genome, a microbial genome, a gene, such as an oncogene, or of a genome of a pathogen.

The second target nucleic acid may also be a mutated nucleic acid sequence, for example a single nucleotide polymorphism (SNP).

The mutant Cpf1 endonuclease used in the method for detection of a second target nucleic acid in a sample may be any of the mutants described herein above, in particular in the section “Mutant Cpf1”.

The mutant Cpf1 endonuclease used in the method for detection of a second target nucleic acid in a sample may be a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions in position 918 and 1013 of SEQ ID NO: 2.

For example mutant Cpf1 endonuclease used in the method for detection of a second target nucleic acid in a sample is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to an amino acid having an uncharged side chain in position 918 and 1013 of SEQ ID NO: 2.

For example mutant Cpf1 endonuclease used in the method for detection of a second target nucleic acid in a sample is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to an amino acid having a non-polar side chain in position 918 and 1013 of SEQ ID NO: 2.

For example mutant Cpf1 endonuclease used in the method for detection of a second target nucleic acid in a sample is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to a glycine, alanine, valine, leucine, isoleucine, serine or threonine in position 918 and 1013 of SEQ ID NO: 2.

For example mutant Cpf1 endonuclease used in the method for detection of a second target nucleic acid in a sample is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to a glycine in position 918 and 1013 of SEQ ID NO: 2.

For example mutant Cpf1 endonuclease used in the method for detection of a second target nucleic acid in a sample is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises the amino acid substitutions R918G and K1013G.

For example mutant Cpf1 endonuclease used in the method for detection of a second target nucleic acid in a sample is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as about 100% sequence identity to SEQ ID NO: 4.

For example mutant Cpf1 endonuclease used in the method for detection of a second target nucleic acid in a sample is a Cpf1 endonuclease or orthologue thereof comprises or consists of SEQ ID NO: 4.

The mutant Cpf1 endonuclease used in the method for detection of a second target nucleic acid in a sample may also comprise other amino acid modifications.

Use of a crRNA-Cpf1 Endonuclease Complex for Diagnosis of a Disease

The present disclosure also relates to methods for diagnosis of any disease which is associated with increased/reduced gene expression and/or with the presence of exogenous genetic material.

One aspect of the present disclosure relates to an in vitro method for diagnosis of a disease in a subject, the method comprising:
a. Providing a crRNA-Cpf1 complex, wherein the Cpf1 is a mutant Cpf1 endonuclease or orthologue thereof as defined herein, and wherein the crRNA is specific for a second target nucleic acid;
b. Providing a labelled ssDNA, wherein the ssDNA is labelled with at least one set of interactive labels comprising at least one fluorophore and at least one quencher;
c. Providing a sample from the subject wherein said sample comprises or is suspected of comprising the second target nucleic acid; and
d. Determining the level and/or concentration of the second target nucleic acid according to the method of detection described herein,
   wherein the second target nucleic acid is a nucleic acid fragment that correlates with the disease,
   thereby diagnosing a disease in a subject.

The method for diagnosis of a disease in a subject may further comprise the step treating said disease. For example, the method may further comprise treating said disease by administering a therapeutically effective agent.

The mutant Cpf1 endonuclease used in the method for detection of a second target nucleic acid in a sample may be any of the mutants described herein above, in particular in the section “Mutant Cpf1”.

Use of a crRNA-Cpf1 Endonuclease Complex for Diagnosis of an Infectious Disease

One aspect of the present disclosure relates to an in vitro method for diagnosis of an infectious disease in a subject, the method comprising:
a. Providing a crRNA-Cpf1 complex, wherein the Cpf1 is a mutant Cpf1 endonuclease or orthologue thereof as defined herein, and wherein the crRNA is specific for a second target nucleic acid;
b. Providing a labelled ssDNA, wherein the ssDNA is labelled with at least one set of interactive labels and wherein said interactive label gives a signal upon cleavage of the ssDNA;
c. Providing a sample from the subject, wherein said sample comprises or is suspected of comprising the second target nucleic acid; and
d. Determining the level and/or concentration of the second target nucleic acid according to the method of detection described herein,
wherein the second target nucleic acid is a nucleic acid fragment of the genome of an infectious agent causing the disease,
thereby diagnosing an infectious disease in a subject.

One aspect of the present disclosure relates to an in vitro method for diagnosis of an infectious disease in a subject, the method comprising:
a. Providing a crRNA-Cpf1 complex, wherein the Cpf1 is a mutant Cpf1 endonuclease or orthologue thereof as defined herein, and wherein the crRNA is specific for a second target nucleic acid;
b. Providing a labelled ssDNA, wherein the ssDNA is labelled with at least one set of interactive labels comprising at least one fluorophore and at least one quencher;
c. Providing a sample from the subject, wherein said sample comprises or is suspected of comprising the second target nucleic acid; and
d. Determining the level and/or concentration of the second target nucleic acid according to the method of detection described herein,
wherein the second target nucleic acid is a nucleic acid fragment of the genome of an infectious agent causing the disease,
thereby diagnosing an infectious disease in a subject.

The interactive label may for example comprise a luminescent label.

The method for diagnosis of an infectious disease in a subject may further comprise the step treating said infectious disease. For example, the method may further comprise treating said infectious disease by administering a therapeutically effective agent.

The method for diagnosis of an infectious disease in a subject may further comprise the step of comparing the level and/or concentration of said second target nucleic acid with a cut-off value,

wherein said cut-off value is determined from the concentration range of said second target nucleic acid in healthy subjects, such as subjects who do not present with the infectious disease,
wherein a level and/or concentration that is greater than the cut-off value indicates the presence of the infectious disease.

An infectious disease is any disease caused by an infectious agent such as viruses, viroids, prions, bacteria, nematodes, parasitic roundworms, pinworms, arthropods, fungi, ringworm and macroparasites.

Thus, the second target nucleic acid may be a genome or fragment thereof of an infectious agent selected from the group consisting of viruses, viroids, prions, bacteria, nematodes, parasitic roundworms, pinworms, arthropods, fungi, ringworm and macroparasites.

The method disclosed herein may be used to diagnose an infection disease in a human.

Thus, the sample comprising the second target nucleic acid may by a sample taken from a human body. For example, the sample may be a human body fluid selected from the group consisting of blood, whole blood, plasma, serum, urine, saliva, tears, cerebrospinal fluid and semen.

The mutant Cpf1 endonuclease used in the method for detection of a second target nucleic acid in a sample may be any of the mutants described herein above, in particular in the section “Mutant Cpf1”.

In one embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises an amino acid substitution at position 1025 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a deletion of one or more amino acid residues of the REC domain defined by residues 298 to 309 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a deletion of one or more amino acid residues of the Lid domain defined by residues 324 to 336 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a deletion of one or more amino acid residues of the Lid domain defined by residues 1006 to 1018 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises a substitution of two or more amino acid residues of the Lid domain defined by residues 1006 to 1018 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises an amino acid substitution at one or more of positions 918, 1013, 1014, 1025 and 1028 of SEQ ID NO: 2.

In another embodiment the present disclosure relates to the use of a crRNA-Cpf1 complex for introducing a single strand break in a specific recognition nucleotide sequence of the first target nucleic acid, wherein the first target nucleic acid and the second target nucleic acid are identical, and wherein the Cpf1 endonuclease or orthologue thereof comprises an amino acid substitution at two or more of positions 918, 1013, 1014, 1025 and 1028 of SEQ ID NO: 2.

The mutant Cpf1 endonuclease used in the method for diagnosis of an infectious disease is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions in position 918 and 1013 of SEQ ID NO: 2.

For example mutant Cpf1 endonuclease used in the method for diagnosis of an infectious disease is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to an amino acid having an uncharged side chain in position 918 and 1013 of SEQ ID NO: 2.

For example mutant Cpf1 endonuclease used in the method for diagnosis of an infectious disease is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to an amino acid having a non-polar side chain in position 918 and 1013 of SEQ ID NO: 2.

For example mutant Cpf1 endonuclease used in the method for diagnosis of an infectious disease is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to a glycine, alanine, valine, leucine, isoleucine, serine or threonine in position 918 and 1013 of SEQ ID NO: 2.

For example mutant Cpf1 endonuclease used in the method for diagnosis of an infectious disease in a sample is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises at least two amino acid substitutions of an amino acid having a charged side chain to a glycine in position 918 and 1013 of SEQ ID NO: 2.

For example mutant Cpf1 endonuclease used in the method for diagnosis of an infectious disease is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity to SEQ ID NO: 2, wherein said polypeptide sequence comprises the amino acid substitutions R918G and K1013G.

For example mutant Cpf1 endonuclease used in the method for diagnosis of an infectious disease is a Cpf1 endonuclease or orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as about 100% sequence identity to SEQ ID NO: 4.

For example mutant Cpf1 endonuclease used in the method for diagnosis of an infectious disease is a Cpf1 endonuclease or orthologue thereof comprises or consists of SEQ ID NO: 4.

The mutant Cpf1 endonuclease used in the method for diagnosis of an infectious disease may also comprise other amino acid modifications.

### Items

1. A mutant Cpf1 endonuclease or orthologue thereof comprising a polypeptide sequence having at least 95% sequence identity to:
   i) the sequence corresponding to residues 1 to 297, 310 to 323, 337 to 1005, and 1019 to 1329 of SEQ ID NO: 2, wherein said polypeptide sequence further comprises at least one amino acid substitution or deletion in the finger domain (residues 298 to 309; SEQ ID NO: 12), the REC domain (residues 324 to 336; SEQ ID NO: 16) or the Lid domain (residues 1006 to 1018; SEQ ID NO: 20) compared to SEQ ID NO: 2; and/or
   i) SEQ ID NO: 2, wherein said polypeptide sequence comprises at least one amino acid substitution in position 918, 1013, 1014, 1025 or 1028 of SEQ ID NO: 2.
2. A mutant Cpf1 endonuclease or orthologue thereof comprising a polypeptide sequence having at least 95% sequence identity to:
   i) the sequence corresponding to residues 1 to 323, 337 to 1005, and 1019 to 1329 of SEQ ID NO: 2, wherein said polypeptide sequence further comprises:
      a. at least two amino acid mutations in the REC domain (residues 324 to 336; SEQ ID NO: 16) compared to SEQ ID NO: 2, wherein each mutation independently is an amino acid substitution or deletion; and/or
      b. at least two amino acid substitutions in the Lid domain (residues 1006 to 1018; SEQ ID NO: 20) compared to SEQ ID NO: 2, wherein at least one of the residues at positions 917 and 1006 is a glutamic acid (E) or an aspartic acid (D);
   and/or
   ii) SEQ ID NO: 2, wherein said polypeptide sequence comprises at least two amino acid substitutions in two positions independently selected from the positions corresponding to residues 918, 1013, 1014, 1025 and 1028 of SEQ ID NO: 2.
3. The mutant Cpf1 endonuclease or orthologue thereof according to item 1, wherein the Cpf1 endonuclease is derived from Francisella novicida.
4. The mutant Cpf1 endonuclease or orthologue thereof according to any one of the preceding items, wherein said mutant Cpf1 comprises a polypeptide sequence having at least 95% sequence identity to the sequence corresponding to residues 1 to 323, 337 to 1005, and 1019 to 1329 of SEQ ID NO: 2, wherein said polypeptide sequence further comprises:
   a. at least two amino acid substitutions or deletions in the REC domain (residues 324 to 336; SEQ ID NO: 16) compared to SEQ ID NO: 2; and/or
   b. at least two amino acid substitutions in the Lid domain (residues 1006 to 1018; SEQ ID NO: 20) compared to SEQ ID NO: 2, with the proviso that at least one of the residues at positions 917 and 1006 of SEQ ID NO: 2 is a glutamic acid (E) or an aspartic acid (D);
   wherein said polypeptide sequence further comprises at least one amino acid substitution in position 918, 1013, 1014, 1025 or 1028 of SEQ ID NO: 2.
5. The mutant Cpf1 endonuclease or orthologue thereof according to any one of the preceding items, wherein one of the at least two amino acid substitutions is a substitution of an amino acid having a charged side chain to an amino acid having an uncharged side chain.
6. The mutant Cpf1 endonuclease or orthologue thereof according to any one of the preceding items, wherein the at least one amino acid substitution is a substitution of an amino acid having a charged side chain to an amino acid having an uncharged side chain.
7. The mutant Cpf1 endonuclease or orthologue thereof according to any one of the preceding items, wherein the at least one amino acid substitution is a substitution of an amino acid having a charged side chain to an amino acid residue having a non-polar side chain.
8. The mutant Cpf1 endonuclease or orthologue thereof according to any one of the preceding items, wherein one of the at least two amino acid substitutions is a substitution of an amino acid having a charged side chain to an amino acid residue having a non-polar side chain.
9. The mutant Cpf1 endonuclease or orthologue thereof according to any one of the preceding items, wherein one of the at least two amino acid substitutions is a substitution of an amino acid having a charged side chain to a glycine, alanine, valine, leucine, isoleucine, serine or threonine.
10. The mutant Cpf1 endonuclease or orthologue thereof according to any one of the preceding items, wherein the at least one amino acid substitution is a substitution of an amino acid having a charged side chain to a glycine, alanine, valine, leucine, isoleucine, serine or threonine.
11. The mutant Cpf1 endonuclease or orthologue thereof according to any one of the preceding items, wherein one of the at least two amino acid substitutions is a substitution of an amino acid having a charged side chain to a glycine.
12. The mutant Cpf1 endonuclease or orthologue thereof according to any one of the preceding items, wherein one of the at least two amino acid substitutions is a substitution of an amino acid having a charged side chain to a glycine.
13. The mutant Cpf1 endonuclease or orthologue thereof according to any one of the preceding items, wherein the at least one amino acid substitution or deletion is in the REC domain.
14. The mutant Cpf1 endonuclease or orthologue thereof according to any one of the preceding items, wherein the at least one amino acid substitution or deletion is a substitution or deletion of at least one residue in the REC domain, such as a substitution or deletion of at least 2 residues, such as a substitution or deletion of at least 3 residues, such as a substitution or deletion of at least 4 residues, such as a substitution or deletion of at least 5 residues, such as a substitution or deletion of at least 6 residues, such as a substitution or deletion of at least 7 residues, such as a substitution or deletion of at least 8 residues, such as a substitution or deletion of at least 9 residues, such as a substitution or deletion of at least 10 residues, such as a substitution or deletion of at least 11 residues, such as a substitution or deletion of at least 12 residues, such as a substitution or deletion of at least 13 residues of the REC domain.
15. The mutant Cpf1 endonuclease or orthologue thereof according to any one of the preceding items, wherein the at least two amino acid substitutions or deletions are a substitution or deletion of at least 2 residues in the REC domain, such as a substitution or deletion of at least 3 residues, such as a substitution or deletion of at least 4 residues, such as a substitution or deletion of at least 5 residues, such as a substitution or deletion of at least 6 residues, such as a substitution or deletion of at least 7 residues, such as a substitution or deletion of at least 8 residues, such as a substitution or deletion of at least 9 residues, such as a substitution or deletion of at least 10 residues, such as a substitution or deletion of at least 11 residues, such as a substitution or deletion of at least 12 residues, such as a substitution or deletion of at least 13 residues of the REC domain.
16. The mutant Cpf1 endonuclease or orthologue thereof according to any one of the preceding items, wherein the at least one substitution or deletion is a substitution or deletion of all the residues of the REC domain.
17. The mutant Cpf1 endonuclease or orthologue thereof according to any one of the preceding items, wherein the at least two substitutions or deletions are a substitution or deletion of all the residues of the REC domain.
18. The mutant Cpf1 endonuclease or orthologue thereof according to any one of the preceding items, wherein the at least one amino acid substitution or deletion is in the Lid domain.
19. The mutant Cpf1 endonuclease or orthologue thereof according to any one of the preceding items, wherein the at least two amino acid substitutions are in the Lid domain.
20. The mutant Cpf1 endonuclease or orthologue thereof according to any one of the preceding items, wherein the at least one amino acid substitution or deletion is a substitution or deletion of at least one residue in the Lid domain, such as a substitution or deletion of at least 2 residues, such as a substitution or deletion of at least 3 residues, such as a substitution or deletion of at least 4 residues, such as a substitution or deletion of at least 5 residues, such as a substitution or deletion of at least 6 residues, such as a substitution or deletion of at least 7 residues, such as a substitution or deletion of at least 8 residues, such as a substitution or deletion of at least 9 residues, such as a substitution or deletion of at least 10 residues, such as a substitution or deletion of at least 11 residues, such as a substitution or deletion of at least 12 residues, such as a substitution or deletion of at least 13 residues of the Lid domain.
21. The mutant Cpf1 endonuclease or orthologue thereof according to any one of the preceding items, wherein the at least two amino acid substitutions area substitution of at least two residues in the Lid domain, such as a substitution or deletion of at least 3 residues, such as a substitution or deletion of at least 4 residues, such as a substitution or deletion of at least 5 residues, such as a substitution or deletion of at least 6 residues, such as a substitution or deletion of at least 7 residues, such as a substitution or deletion of at least 8 residues, such as a substitution or deletion of at least 9 residues, such as a substitution or deletion of at least 10 residues, such as a substitution or deletion of at least 11 residues, such as a substitution or deletion of at least 12 residues, such as a substitution or deletion of at least 13 residues of the Lid domain.
22. The mutant Cpf1 endonuclease or orthologue thereof according to any one of the preceding items, wherein the at least one substitution or deletion is a substitution or deletion of all the residues of the Lid domain.
23. The mutant Cpf1 endonuclease or orthologue thereof according to any one of the preceding items, wherein the at least two substitutions are a substitution of all the residues of the Lid domain.
24. The mutant Cpf1 endonuclease or orthologue thereof according to any one of the preceding items, wherein the at least one amino acid substitution or deletion is in the finger domain.
25. The mutant Cpf1 endonuclease or orthologue thereof according to any one of the preceding items, wherein the at least one amino acid substitution or deletion is a substitution or deletion of at least one residue in the finger domain, such as a substitution or deletion of at least 2 residues, such as a substitution or deletion of at least 3 residues, such as a substitution or deletion of at least 4 residues, such as a substitution or deletion of at least 5 residues, such as a substitution or deletion of at least 6 residues, such as a substitution or deletion of at least 7 residues, such as a substitution or deletion of at least 8 residues, such as a substitution or deletion of at least 9 residues, such as a substitution or deletion of at least 10 residues, such as a substitution or deletion of at least 11 residues, such as a substitution or deletion of at least 12 residues of the finger domain.
26. The mutant Cpf1 endonuclease or orthologue thereof according to any one of the preceding items, wherein the at least one substitution or deletion is a substitution or deletion of all the residues of the finger domain.
27. The mutant Cpf1 endonuclease or orthologue thereof according to any one of the preceding items, wherein the at least one amino acid substitution is R918G, K1013G, R1014G, Q1025G or E1028G.
28. The mutant Cpf1 endonuclease or orthologue thereof according to any one of the preceding items, wherein the at least two amino acid substitutions are independently selected from R918G, K1013G, R1014G, Q1025G and E1028G.
29. The mutant Cpf1 endonuclease or orthologue thereof according to any one of the preceding items, wherein the Cpf1 endonuclease is a nicking endonuclease.
30. The mutant Cpf1 endonuclease or orthologue thereof according to any one of the preceding items, wherein said amino acid substitution is at position K1013.
31. The mutant Cpf1 endonuclease or orthologue thereof according to any one of the preceding items, comprising substitution at position corresponding to K1013 of SEQ ID NO: 2.
32. The mutant Cpf1 endonuclease or orthologue thereof according to any one of the preceding items, wherein said amino acid substitution is at positions R918 and K1013.
33. The mutant Cpf1 endonuclease or orthologue thereof according to any one of the preceding items, comprising substitutions at positions corresponding to R918 and K1013 of SEQ ID NO: 2.
34. The mutant Cpf1 endonuclease or orthologue thereof according to any one of the preceding items, wherein said amino acid substitution is at positions K1013 and R1014.
35. The mutant Cpf1 endonuclease or orthologue thereof according to any one of the preceding items, comprising substitutions at positions corresponding to K1013 and R1014 of SEQ ID NO: 2.
36. The mutant Cpf1 endonuclease or orthologue thereof according to any one of the preceding items, wherein said amino acid substitution is K1013G.
37. The mutant Cpf1 endonuclease or orthologue thereof according to any one of the preceding items, comprising substitution corresponding to K1013G of SEQ ID NO: 2.
38. The mutant Cpf1 endonuclease or orthologue thereof according to any one of the preceding items, wherein the Cpf1 endonuclease comprises the amino acid substitutions K1013G and R1014G.
39. The mutant Cpf1 endonuclease or orthologue thereof according to any one of the preceding items, comprising substitutions corresponding to K1013G and R1014G of SEQ ID NO: 2.
40. The mutant Cpf1 endonuclease or orthologue thereof according to any one of the preceding items, wherein said amino acid substitution is at position Q1025.
41. The mutant Cpf1 endonuclease or orthologue thereof according to any one of the preceding items, comprising substitution at position corresponding to Q1025 of SEQ ID NO: 2.
42. The mutant Cpf1 endonuclease or orthologue thereof according to any one of the preceding items, wherein said amino acid substitution is at position E1028.
43. The mutant Cpf1 endonuclease or orthologue thereof according to any one of the preceding items, comprising substitution at position corresponding to E1028 of SEQ ID NO: 2.
44. The mutant Cpf1 endonuclease or orthologue thereof according to any one of the preceding items, wherein said amino acid substitution is at positions Q1025 and E1028.
45. The mutant Cpf1 endonuclease or orthologue thereof according to any one of the preceding items, comprising substitutions at positions corresponding to Q1025 and E1028 of SEQ ID NO: 2.
46. The mutant Cpf1 endonuclease or orthologue thereof according to any one of the preceding items, wherein said amino acid substitution is Q1025G.
47. The mutant Cpf1 endonuclease or orthologue thereof according to any one of the preceding items, comprising substitution corresponding to Q1025G of SEQ ID NO: 2.
48. The mutant Cpf1 endonuclease or orthologue thereof according to any one of the preceding items, wherein said amino acid substitution is E1028G.
49. The mutant Cpf1 endonuclease or orthologue thereof according to any one of the preceding items, comprising substitution corresponding to E1028G of SEQ ID NO: 2.
50. The mutant Cpf1 endonuclease or orthologue thereof according to any one of the preceding items, wherein the Cpf1 endonuclease comprises the amino acid substitutions Q1025G and E1028G.
51. The mutant Cpf1 endonuclease or orthologue thereof according to any one of the preceding items, comprising substitutions corresponding to Q1025G and E1028G of SEQ ID NO: 2.
52. The mutant Cpf1 endonuclease or orthologue thereof according to any one of the preceding items, wherein the Cpf1 endonuclease comprises at least two amino acid substitutions or deletions in the REC domain (residues 324 to 336; SEQ ID NO: 16) compared to SEQ ID NO: 2, and at least one amino acid substitution in any one of positions 918, 1013, 1014, 1025 and 1028 of SEQ ID NO: 2.
53. The mutant Cpf1 endonuclease or orthologue thereof according to any one of the preceding items, wherein the Cpf1 endonuclease comprises at least two amino acid substitutions in the Lid domain (residues 1006 to 1018; SEQ ID NO: 20) compared to SEQ ID NO: 2, and at least one amino acid substitution in any one of positions 918, 1013, 1014, 1025 and 1028 of SEQ ID NO: 2.
54. The mutant Cpf1 endonuclease or orthologue thereof according to any one of the preceding items, wherein the Cpf1 endonuclease comprises at least two amino acid substitutions or deletions in the REC domain (residues 324 to 336; SEQ ID NO: 16) compared to SEQ ID NO: 2, and at least two amino acid substitutions in any one of positions 918, 1013, 1014, 1025 and 1028 of SEQ ID NO: 2.
55. The mutant Cpf1 endonuclease or orthologue thereof according to any one of the preceding items, wherein the Cpf1 endonuclease comprises at least two amino acid substitutions in the Lid domain (residues 1006 to 1018; SEQ ID NO: 20) compared to SEQ ID NO: 2, and at least two amino acid substitutions in any one of positions 918, 1013, 1014, 1025 and 1028 of SEQ ID NO: 2.
56. The mutant Cpf1 endonuclease or orthologue thereof according to any one of the preceding items, wherein the Cpf1 endonuclease comprises or consists of a polypeptide having at least 95% sequence identity to SEQ ID NO: 3 or SEQ ID NO: 4.
57. The mutant Cpf1 endonuclease or orthologue thereof according to any one of the preceding items, wherein the Cpf1 endonuclease comprises or consists of a polypeptide of SEQ ID NO: 3 or SEQ ID NO: 4.
58. The mutant Cpf1 endonuclease or orthologue thereof according to any one of the preceding items, wherein the REC domain of the mutant Cpf1 endonuclease comprises or consists of SEQ ID NO: 14.
59. The mutant Cpf1 endonuclease or orthologue thereof according to any one of the preceding items, wherein the Lid domain of the mutant Cpf1 endonuclease comprises or consists of SEQ ID NO: 18.
60. The mutant Cpf1 endonuclease or orthologue thereof according to any one of the preceding items, wherein the finger domain of the mutant Cpf1 endonuclease comprises or consists of SEQ ID NO: 22.
61. The mutant Cpf1 endonuclease or orthologue thereof according to any one of the preceding items, wherein the mutant Cpf1 has one or more altered activity compared to the wild-type Cpf1, said activity being selected from the group consisting of: double-stranded cleavage of a target or non-target nucleic acid; single-stranded cleavage of stranded target or non-target nucleic acid; single-stranded cleavage of a nucleic acid in a non-specific manner, optionally enhanced by single-stranded or double-stranded target DNA.
62. The mutant Cpf1 endonuclease or orthologue thereof according to any one of the preceding items, wherein the residue at position 917 of SEQ ID NO: 2 is a glutamic acid (E) or an aspartic acid (D).
63. The mutant Cpf1 endonuclease or orthologue thereof according to any one of the preceding items, wherein the residue at position 1006 of SEQ ID NO: 2 is a glutamic acid (E) or an aspartic acid (D).
64. A polynucleotide encoding the mutant Cpf1 endonuclease or orthologue thereof according to any one of the preceding items.
65. The polynucleotide according to item 64, wherein said polynucleotide is codon-optimized for expression in a host cell.
66. A recombinant vector comprising a polynucleotide according to any one of items 1 to 64, or a nucleic acid sequence encoding a mutant Cpf1 endonuclease or orthologue thereof according to any one of items 1 to 64.
67. The recombinant vector according to item 66, wherein said polynucleotide or nucleic acid sequence is operably linked to a promoter.
68. The recombinant vector according to any one of items 66 to 67, further comprising a nucleic acid sequence encoding a guide RNA (crRNA) operably linked to a promoter, wherein the crRNA binds the encoded Cpf1 endonuclease and a fragment with sufficient base pairs to hybridize to a target nucleic acid.
69. The recombinant vector according to any one of items 66 to 68, wherein the crRNA consists of a constant region of 19 or 20 nucleotides, and a variable region consisting of between 12 and 24 nucleotides, such that said crRNA is at least 40 nucleotides in length, such as 41 nucleotides in length, such as 42 nucleotides in length, such as 43 nucleotides in length, such as 44 nucleotides in length.
70. The recombinant vector according to any one of items 66 to 69, wherein the constant region of the crRNA is as set out in SEQ ID NO: 5.
71. A cell capable of expressing the mutant Cpf1 or orthologue thereof according to any one of items 1 to 63, the polynucleotide according to any one of items 64 to 65, or the recombinant vector according to any one of items 66 to 70.
72. A system for expression of a crRNA-Cpf1 complex comprising
   a. a polynucleotide according to any one of items 64 to 65, or a recombinant vector according to any one of items 66 to 70 comprising a polynucleotide encoding a mutant Cpf1 endonuclease or orthologue thereof;
   b. a polynucleotide or a recombinant vector comprising a polynucleotide encoding a guide RNA (crRNA), optionally operably linked to a promoter.
73. The system according to item 72, further comprising
   c. a cell for expression of the polynucleotide or the recombinant vector of a. and b. above.
74. The cell according to item 71 or the system according to any one of items 72 to 73, wherein said cell is a prokaryotic or a eukaryotic cell.
75. Use of a crRNA-Cpf1 complex for introducing a single strand break in a first target nucleic acid, wherein:
   a. a Cpf1 endonuclease or orthologue thereof is contacted with a guide RNA (crRNA), thereby obtaining a crRNA-Cpf1 complex capable of recognising a second target nucleic acid, the second target nucleic acid comprising a protospacer adjacent motif (PAM), and
   b. the crRNA-Cpf1 complex is contacted with the first target nucleic acid;
   whereby a single strand break is made in the first target sequence.
76. The use according to item 75, wherein the Cpf1 endonuclease or orthologue thereof is according to any one of items 1 to 63, or encoded by a polynucleotide or a vector according to any one of items 64 to 70.
77. The use according to any one of items 75 to 76, wherein the crRNA hybridizes to the second target nucleic acid.
78. The use according to any one of items 75 to 77, wherein hybridization of the crRNA to the second target nucleic acid activates the crRNA-Cpf1 complex.
79. The use according to any one of items 75 to 78, wherein the single strand break is made in a specific recognition nucleotide sequence of the first target nucleic acid.
80. The use according to any one of items 75 to 79, wherein the single strand break is made at one or more nonspecific nucleotide sequences of the first target nucleic acid.
81. The use according to any one of items 75 to 80, wherein the crRNA is as defined in any one of the preceding items.
82. The use according to any one of items 75 to 81, wherein the molar ratio of Cpf1 to crRNA is between 0.5:3.0 and 1.0:1.0, such as 0.7:2.5, such as 0.8:2.0, such as 0.9:1.75, such as 0.95:1.5, such as 1.0:1.4, such as 1.0:1.3, such as 1.1:1.2.
83. The use according to any one of items 75 to 82, wherein the molar ratio of Cpf1:crRNA:second target nucleic acid is 0.5:3.0:5.0, such as 0.7:2.5:4.0, such as 0.8:2.0:3.5, such as 0.9:1.75:3.0, such as 0.95:1.5:2.0, such as 1.0:1.4:1.9, such as 1.0:1.3:1.7.
84. The use according to any one of items 75 to 83, wherein the second target nucleic acid comprises or consists of a recognition sequence comprising a sequence of at least 15 consecutive nucleotides, such as at least 16 consecutive nucleotides, such as at least 17 consecutive nucleotides, such as at least 18 consecutive nucleotides, such as at least 19 consecutive nucleotides, such as at least 20 consecutive nucleotides, such as at least 21 consecutive nucleotides, such as at least 22 consecutive nucleotides, such as at least 23 consecutive nucleotides, such as at least 24 consecutive nucleotides, such as at least 25 consecutive nucleotides, such as at least 26 consecutive nucleotides, such as at least 27 consecutive nucleotides, with the proviso that the 3 nucleic acids at the 5′-end consist of a PAM sequence.
85. The use according to any one of items 75 to 84, wherein the PAM comprises or consists of the sequence 5′-YRN-3′ or 5′-RYN-3′.
86. The use according to any one of items 75 to 85, wherein the PAM comprises or consists of the sequence 5′-TTN-3′.
87. The use according to any one of items 75 to 86, wherein the first target nucleic acid and the second target nucleic acid are DNA or RNA.
88. The use according to any one of items 75 to 87, wherein the second target nucleic acid is double stranded DNA.
89. The use according to any one of items 75 to 88, wherein the second target nucleic acid is DNA selected from the group consisting of genomic DNA, chromatin, nucleosomes, plasmid DNA, methylated DNA, synthetic DNA, and DNA fragments.
90. The use according to any one of items 75 to 89, wherein the second target nucleic acid is RNA.
91. The use according to any one of items 75 to 90, wherein the first target nucleic acid and the second target nucleic acid are identical.
92. The use according to any one of items 75 to 91, wherein the first target nucleic acid and the second target nucleic acid are the same molecule, and wherein the Cpf1 endonuclease or orthologue thereof comprises amino acid substitutions at position 1013 and 1014 of SEQ ID NO: 2.
93. The use according to any one of items 75 to 92 wherein the first target nucleic acid and the second target nucleic acid are the same molecule, and wherein the Cpf1 endonuclease or orthologue thereof comprises or consists of SEQ ID NO: 3.
94. The use according to any one of items 75 to 93, wherein the single strand break is made in a specific recognition nucleotide sequence of the first target nucleic acid, and wherein the Cpf1 endonuclease or orthologue thereof comprises amino acid substitutions at position 1013 and 1014 of SEQ ID NO: 2.
95. The use according to any one of items 75 to 94, wherein the Cpf1 performs the single strand break in the first target nucleic acid specifically or non-specifically, and wherein the Cpf1 endonuclease or orthologue thereof comprises or consists of SEQ ID NO: 3.
96. The use according to any one of items 75 to 95, wherein the first target nucleic acid and the second target nucleic acid are different, and wherein the Cpf1 endonuclease or orthologue thereof comprises amino acid substitutions at position 918 and 1013 of SEQ ID NO: 2.
97. The use according to any one of items 75 to 96, wherein the first target nucleic acid and the second target nucleic acid are different, and wherein the Cpf1 endonuclease or orthologue thereof comprises or consists of SEQ ID NO: 4.
98. The use according to any one of items 75 to 97, wherein the Cpf1 performs the single strand break in the first target nucleic acid specifically or non-specifically, and wherein the Cpf1 endonuclease or orthologue thereof comprises amino acid substitutions at position 918 and 1013 of SEQ ID NO: 2.
99. The use according to any one of items 75 to 98, wherein the Cpf1 performs the single strand break in the first target nucleic acid specifically or non-specifically, and wherein the Cpf1 endonuclease or orthologue thereof comprises or consists of SEQ ID NO: 4.
100. The use according to any one of items 75 to 99, wherein the first target nucleic acid and the second target nucleic acid are different, and wherein the Cpf1 endonuclease or orthologue thereof comprises two or more amino acid substitutions or deletions at any one of positions 324 to 336 of SEQ ID NO: 2, such as wherein the Cpf1 endonuclease or orthologue thereof comprises amino acid substitutions at all of residues corresponding to positions 324 to 336 of SEQ ID NO: 2, such as wherein the Cpf1 endonuclease or orthologue thereof comprises amino acid deletions at all of residues corresponding to positions 324 to 336 of SEQ ID NO: 2.
101. The use according to any one of items 75 to 100, wherein the first target nucleic acid and the second target nucleic acid are different, and wherein the Cpf1 endonuclease or orthologue thereof comprises or consists of SEQ ID NO: 34 or SEQ ID NO: 36.
102. The use according to any one of items 75 to 101, wherein the Cpf1 performs the single strand break in the first target nucleic acid specifically or non-specifically, and wherein the Cpf1 endonuclease or orthologue thereof comprises two or more amino acid substitutions or deletions at any one of positions 324 to 336 of SEQ ID NO: 2, such as wherein the Cpf1 endonuclease or orthologue thereof comprises amino acid substitutions at all of residues corresponding to positions 324 to 336 of SEQ ID NO: 2, such as wherein the Cpf1 endonuclease or orthologue thereof comprises amino acid deletions at all of residues corresponding to positions 324 to 336 of SEQ ID NO: 2.
103. The use according to any one of items 75 to 102, wherein the Cpf1 performs the single strand break in the first target nucleic acid specifically or non-specifically, and wherein the Cpf1 endonuclease or orthologue thereof comprises or consists of SEQ ID NO: 34 or SEQ ID NO: 36.
104. The use according to any one of items 75 to 103, wherein the first target nucleic acid and the second target nucleic acid are different, and wherein the Cpf1 endonuclease or orthologue thereof comprises two or more amino acid substitutions at any one of positions 1006 to 1018 of SEQ ID NO: 2, such as wherein the Cpf1 endonuclease or orthologue thereof comprises amino acid substitutions at all of residues corresponding to positions 1006 to 1018 of SEQ ID NO: 2.
105. The use according to any one of items 75 to 104, wherein the first target nucleic acid and the second target nucleic acid are different, and wherein the Cpf1 endonuclease or orthologue thereof comprises or consists of SEQ ID NO: 38.
106. The use according to any one of items 75 to 105, wherein the Cpf1 performs the single strand break in the first target nucleic acid specifically or non-specifically, and wherein the Cpf1 endonuclease or orthologue thereof comprises two or more amino acid substitutions at any one of positions 1006 to 1018 of SEQ ID NO: 2, such as wherein the Cpf1 endonuclease or orthologue thereof comprises amino acid substitutions at all of residues corresponding to positions 1006 to 1018 of SEQ ID NO: 2.
107. The use according to any one of items 75 to 106, wherein the Cpf1 performs the single strand break in the first target nucleic acid specifically or non-specifically, and wherein the Cpf1 endonuclease or orthologue thereof comprises or consists of SEQ ID NO: 38.
108. The use according to any one of items 75 to 107, wherein the first target nucleic acid and the second target nucleic acid are identical or different, and wherein the Cpf1 endonuclease or orthologue thereof comprises amino acid substitutions at positions corresponding to 1025 and 1028 of SEQ ID NO: 2.
109. The use according to any one of items 75 to 108, wherein the first target nucleic acid and the second target nucleic acid are identical or different, and wherein the Cpf1 endonuclease or orthologue thereof comprises or consists of SEQ ID NO: 32.
110. The use according to any one of items 75 to 109, wherein the Cpf1 performs the single strand break in the first target nucleic acid specifically, wherein the target nucleic acid is in double stranded form, and wherein the Cpf1 endonuclease or orthologue thereof comprises two or more amino acid substitutions at positions corresponding to 1025 and 1028 of SEQ ID NO: 2.
111. The use according to any one of items 75 to 110, wherein the Cpf1 performs the single strand break in the first target nucleic acid specifically, and wherein the Cpf1 endonuclease or orthologue thereof comprises or consists of SEQ ID NO: 32.
112. The use according to any one of items 75 to 111, wherein said method is performed ex-vivo.
113. A method of introducing a single strand break in a first target nucleic acid, comprising the steps of:
   a. designing a guide-RNA (crRNA) capable of recognising a second target nucleic acid comprising a protospacer adjacent motif (PAM);
   b. contacting the crRNA of step a. with a Cpf1 endonuclease or orthologue thereof, thereby obtaining a crRNA-Cpf1 complex capable of binding to said second target nucleic acid, and
   c. contacting the crRNA and the Cpf1 with said first target nucleic acid, thereby introducing one or more single strand breaks in the first target nucleic acid.
114. The method according to item 113, wherein the Cpf1 endonuclease or orthologue thereof is according to any one of items 1 to 63, or encoded by a polynucleotide or a vector according to any one of items 64 to 70.
115. The method according to any one of items 113 to 114, wherein the steps b. and c. can occur simultaneously or one after the other.
116. The method according to any one of items 113 to 115, wherein the method is performed in a cell in vitro.
117. The method according to any one of items 113 to 116, wherein the single strand break is made in a specific recognition nucleotide sequence of the first target nucleic acid.
118. The method according to any one of items 113 to 117, wherein the single strand break is made specifically or non-specifically in the first target nucleic acid.
119. The method according to any one of items 113 to 118, wherein the first and the second target nucleic acids are as defined in any one of the preceding items.
120. An in vitro method of introducing a site-specific, double-stranded break at a second target nucleic acid in a mammalian cell, the method comprising introducing into the mammalian cell a crRNA-Cpf1 complex, wherein the Cpf1 is a mutant Cpf1 endonuclease or orthologue according to any one of items 1 to 63, and wherein the crRNA is specific for the second target nucleic acid.
121. A method for detection of a second target nucleic acid in a sample, the method comprising:
   a. Providing a crRNA-Cpf1 complex, wherein the Cpf1 is a Cpf1 endonuclease or orthologue thereof according to any one of items 1 to 63, and wherein the crRNA is specific for the second target nucleic acid;
   b. Providing a labelled ssDNA, wherein the ssDNA is labelled with at least one set of interactive labels comprising at least one dye and at least one quencher;
   c. Contacting the crRNA-Cpf1 complex and the ssDNA with the sample, wherein the sample comprises at least one second target nucleic acid; and
   d. Detecting cleavage of the ssDNA by detecting a fluorescent signal from the fluorophore, thereby detecting the presence of the second target nucleic acid in the sample.
122. The method according to item 121, wherein step c. comprises activation of the crRNA-Cpf1 complex, such as activation by single-stranded or double-stranded target DNA.
123. The method according to any one of items 121 to 122, further comprising:
   e. determining the level and/or concentration of the second target nucleic acid, wherein the level and/or concentration of the second target nucleic acid is correlated to the cleaved ssDNA.
124. The method according to any one of items 121 to 123, wherein the method can detect a second target nucleic acid at a concentration in the range of nanomolar or below, such as in a range of picomolar or below, such as in a range of femtomolar or below, such as in a range of attomolar or below.
125. The method according to any one of items 121 to 124, wherein the ssDNA is labelled in at least one base in any position along the chain.
126. The method according to any one of items 121 to 125, wherein the at least one dye is a fluorophore.
127. The method according to any one of items 121 to 126, wherein step d. comprises detecting a fluorescent signal resulting from cleavage of the ssDNA.
128. The method according to any one of items 121 to 127, wherein the sample comprises DNA and/or RNA.
129. The method according to any one of items 121 to 128, wherein the sample is suspected of comprising the second target nucleic acid.
130. The method according to any one of items 121 to 129, wherein the second target nucleic acid is a nucleic acid fragment of a viral genome, a microbial genome, a gene, or of a genome of a pathogen.
131. An in vitro method for diagnosis of an infectious disease in a subject, the method comprising:
   a. Providing a crRNA-Cpf1 complex, wherein the Cpf1 is a Cpf1 endonuclease or orthologue thereof according to any one of items 1 to 63, and wherein the crRNA is specific for a second target nucleic acid;
   b. Providing a labelled ssDNA, wherein the ssDNA is labelled with at least one set of interactive labels comprising at least one dye and at least one quencher;
   c. Providing a sample from the subject, wherein said sample comprises or is suspected of comprising the second target nucleic acid; and
   d. Determining the level and/or concentration of the second target nucleic acid as defined in any one of the preceding items,
   wherein the second target nucleic acid is a nucleic acid of the genome of an infectious agent causing the disease or a fragment thereof,
   thereby diagnosing an infectious disease in a subject.
132. The method according to item 131, further comprising the step of treating said infectious disease.
133. The method according to item 132, further comprising treating said infectious disease by administration of a therapeutically effective compound.
134. The method according to any one of items 131 to 133, further comprising the step of comparing the level and/or concentration of said second target nucleic acid with a cut-off value,
   wherein said cut-off value is determined from the concentration range of said second target nucleic acid in healthy subjects, such as subjects who do not present with the infectious disease,
   wherein a level and/or concentration that is greater than the cut-off value indicates the presence of the infectious disease.
135. The method according to any one of items 131 to 134, wherein said infection disease is caused by an infectious agent and wherein the infectious agent comprises viruses, viroids, prions, bacteria, nematodes, parasitic roundworms, pinworms, arthropods, fungi, ringworm and macroparasites.
136. The method according to any one of items 131 to 135, wherein the subject is a human.
137. The method according to any one of items 131 to 136, wherein the sample body fluid selected from the group consisting of blood, whole blood, plasma, serum, urine, saliva, tears, cerebrospinal fluid and semen.
138. The methods according to any one of items 131 to 137, wherein the Cpf1 endonuclease or orthologue thereof comprises amino acid substitutions at position 918 and 1013 of SEQ ID NO: 2.
139. The methods according to any one of items 131 to 138, wherein the Cpf1 endonuclease or orthologue thereof comprises or consists of SEQ ID NO: 4.

### EXAMPLES

### Example 1. Cleavage Assay

The double strand DNA target was prepared by annealing:
the target-strands (t-strand) 5′-atgcagtggccttattaaatgacttctcTAACG-3′-6FAM (SEQ ID NO: 6) and non-target stands (nt-strand) 6FAM-5′-cgTTAgagaagtcatttaataaggccactgcat-3′ (SEQ ID NO: 7) for labelled DNA and
the t-strands 5′-atgcagtggccttattaaatgacttctcTAACG-3′ (SEQ ID NO: 8) and
nt-target stands 5′-cgTTAgagaagtcatttaataaggccactgcat-3′ (SEQ ID NO: 9) for non-labelled DNA target.

The cleavage assay was performed as follows:
4 pmoles of FnCpf1 protein (the wild type protein was used as control) and 5.2 pmoles of crRNA (rArArUrUrUrCrUrArCrUrGrUrUrGrUrArGrArUrCrArCrCrGrArGrUrGrCrArGrCrCrArCrUrCrGr GrCrGrCrU) (SEQ ID NO: 10) were mixed in in 20 mM Bicine-HCl pH8, 150 mM KCl, 0.5 mM TCPE pH8 and 5 mM MgCl at 25° C. for 30 min. Then 6 pmoles of double strand labelled DNA target was added to the mixture and incubate it for other 30 mins at 25° C. The reactions were stopped by adding equal volume of stop solution (8 M Urea and 100 mM EDTA at pH8) followed by incubation at 95° C. for 5 min. The samples were loaded on 15% Novex TBE-Urea Gels (Invitrogen) and run according to the manufacturer instructions. The gel was visualized using an Odyssey FC Imaging System (Li-Cor).

For the non-specific ssDNA the cleavage assay was performed as follows:
4 pmoles of FnCpf1 protein (the wild type protein was used as control) and 5.2 pmoles of crRNA were mixed in in 20 mM Bicine-HCl pH8, 150 mM KCl, 0.5 mM TCPE pH8 and 5 mM MgCl at 25° C. for 30 min. Then 6 pmoles of double strand non-labelled DNA target was added to the mixture and incubate it for other 30 mins at 25° C. Then labelled non-specific ssDNA (/5′ 6-FAM/AGCATGCCCAAATTGCTTACATATGTGTTACGACGGT) (SEQ ID NO: 23) was added and the mixture was incubated at 25° for 30 mins. The reactions were stopped by adding equal volume of stop solution (8 M Urea and 100 mM EDTA at pH8) followed by incubation at 95° C. for 5 min. The samples were loaded on 15% Novex TBE-Urea Gels (Invitrogen) and run according to the manufacture instructions. The gel was visualized using an Odyssey FC Imaging System (Li-Cor).

Endonuclease activity was calculated from the intensity (I) of the DNA bands. The intensity was quantified using ImageStudio. The intensity of the uncleaved DNA (luc) was used as reference. The % of cleavage of NTS (Non-Target Strand), TS (Target Strand) and SSDNA (non-specific single strand) was calculated using the following formula: (100*Iₙ)/I_{uc} where Iₙ is the intensity of the band of interested. The average of at least 3 independent experiments is used in the plot and the standard deviation is represented in the error bars.

### Results:

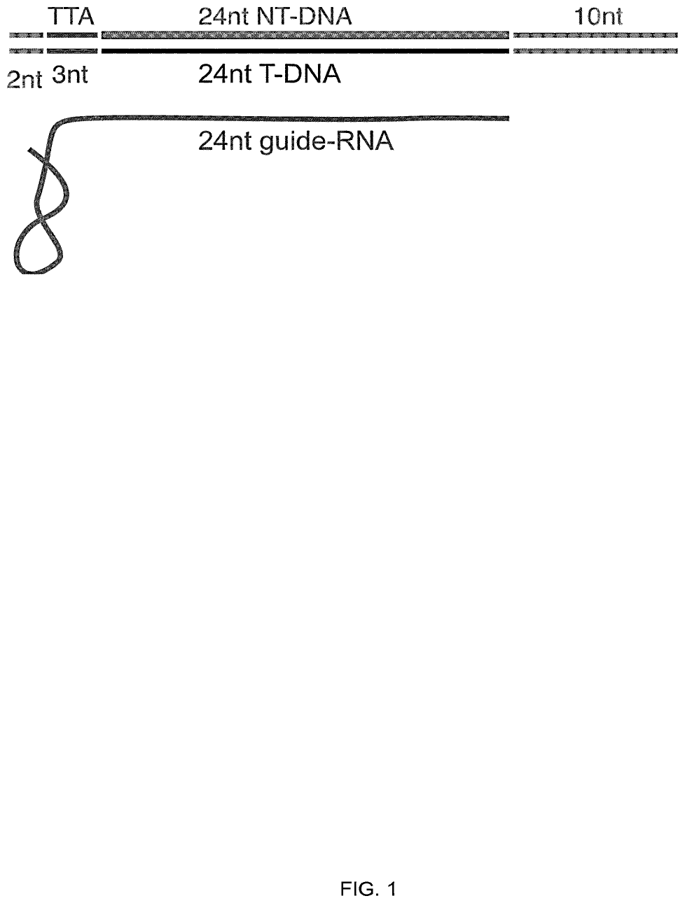

In the DNA duplex contains a PAM sequence (5′-TTA-3′) and a target sequence in the t-strand that is complementary to a part of the crRNA (FIG. 1).

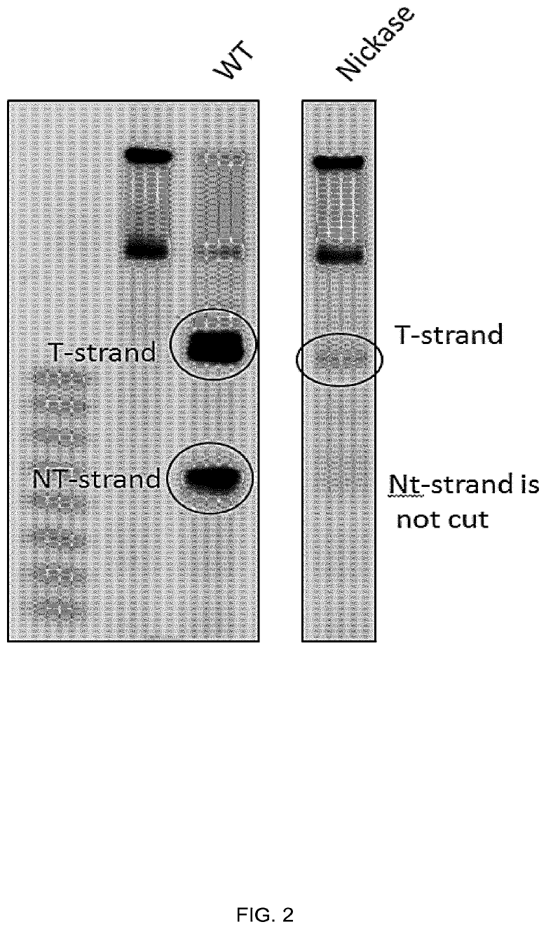

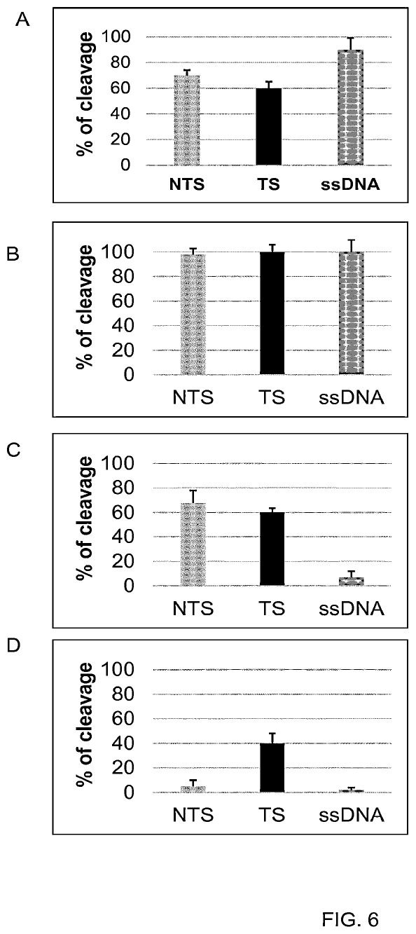

The FnCpf1 wild type protein is able to cut both the t-strand and nt-strand (FIGS. 2-4 and 6). Mutant 1 (SEQ ID NO: 3; K1013-R1014Cpf1) is able to cut only the t-strand (FIGS. 2, 4 and 6D). This mutant is useful as a nickase.

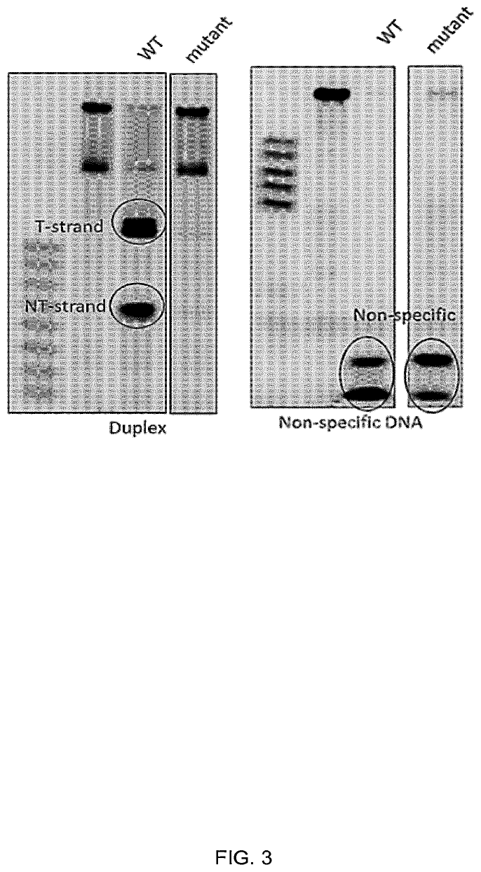

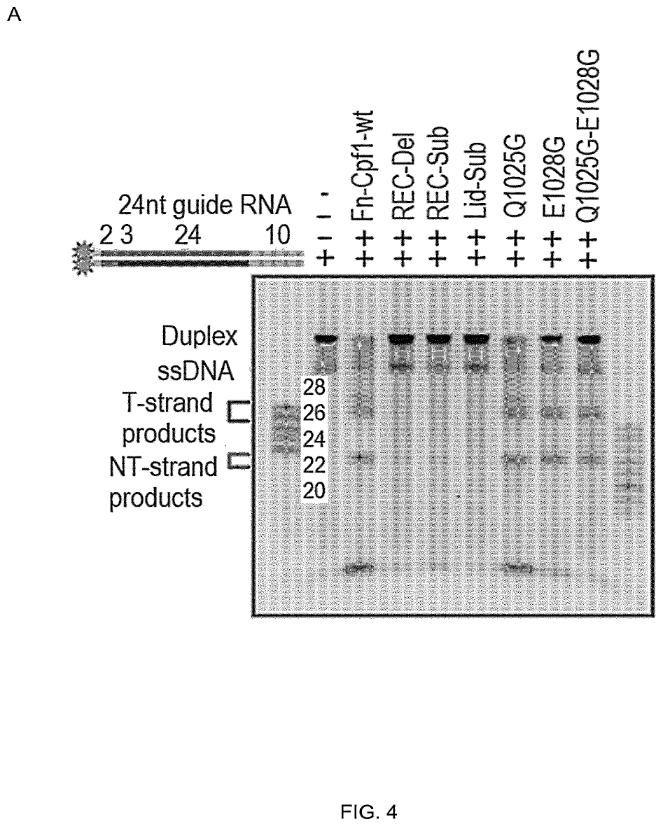

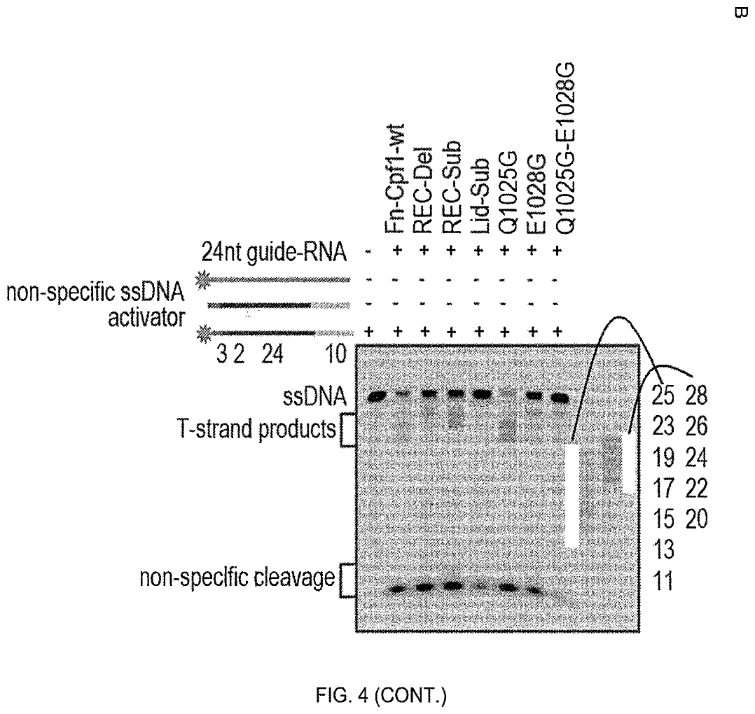

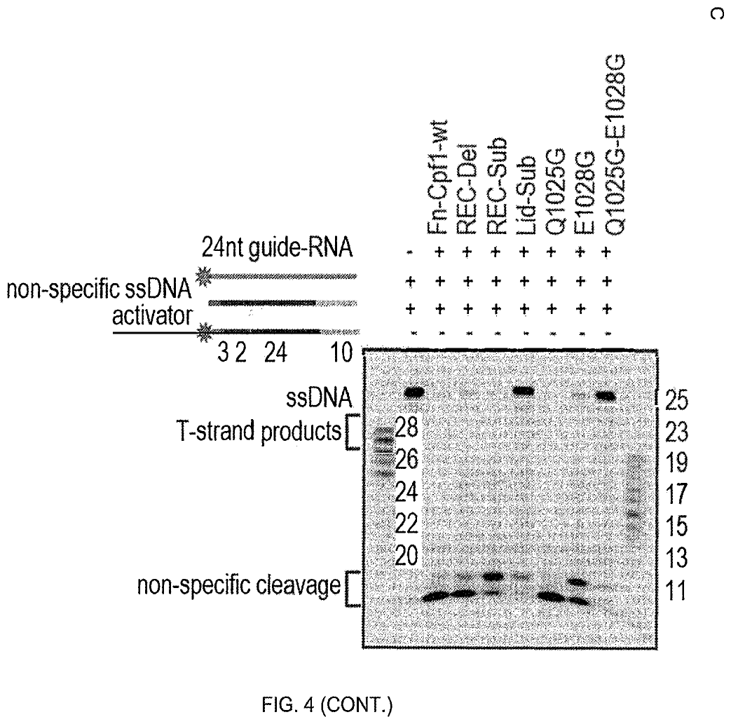

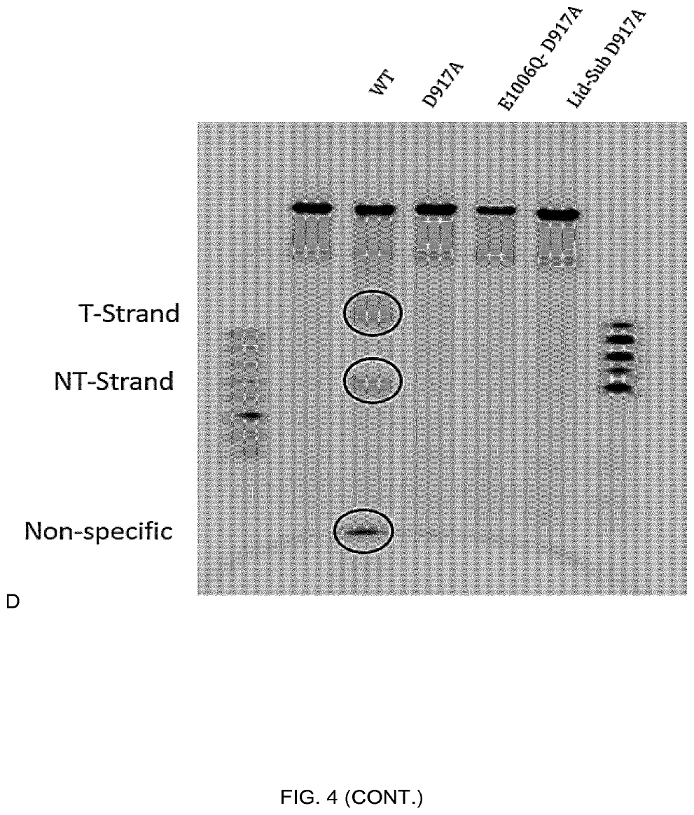

The FnCpf1 wild type protein is able to cut non-specific ssDNA after activation with the double strand DNA-target (FIGS. 3, 4). Mutant 2 (SEQ ID NO: 4; R918G-K1013G) is not able to cut the specific DNA-target, neither as ssDNA nor as ddDNA, as well as the nt-strand, but is able to cut the non-specific ssDNA (FIGS. 3, 4).

The Cpf1 Q1025G mutant (SEQ ID NO: 30) is able to cut both the target-strand and the non-target strand when they are in the double strand DNA form more efficiently than the wild type. The mutant shows increased capacity for cutting the target-strand alone as single strand DNA (FIGS. 4A and B, and 6B). This mutant is able to cut non-specific ssDNA after activation with the double strand DNA-target and single-stranded DNA target. This mutant is useful for genome editing.

The Cpf1 Q1025G E1028G Mutant (SEQ ID NO: 32) is able to cut both the target-strand and non-target strand when they are in the double strand DNA form. The mutant shows a reduced capacity for cutting the target-strand alone as single strand DNA (FIGS. 4A, B, and 6C). This mutant does not seem able to cut non-specific single-stranded DNA after activation with the double strand DNA-target. This mutant can be used for genome editing in a safer way because it does not have non-specific ssDNA activity that can be dangerous for the cells. The presence of non-specific nucleases that induce breaks in ssDNA can cut DNA during duplication, replication and transcription thus possibly generating random breaks in the genome of the cell and therefore inducing undesired mutations, genomic damage and cytotoxicity.

A Cpf1 mutant carrying a deletion of the entire REC domain (SEQ ID NO: 34) is NOT able to cut neither the target-strand nor the non-target strand. The mutant shows a reduced capacity for cutting the target strand alone as single strand DNA (FIG. 4A, B). This mutant is able to cut non-specific ssDNA after activation with the double strand DNA-target. It does not cut the activator so this protein will retain non-specific ssDNA activity longer compared to the wild type.

A Cpf1 mutant carrying a substituted REC domain (SEQ ID NO: 36) is NOT able to cut the target-strand or the non-target-strand when they are in the double strand DNA form. The mutant shows a reduced capacity for cutting the target-strand alone as single strand DNA. The mutant is able to cut non-specific ssDNA after activation with the double strand and single strand DNA-target. It does not cut the activator so this protein will retain non-specific ssDNA activity longer compared to the wild type.

A Cpf1 mutant carrying a substitution of the Lid domain (SEQ ID NO: 38) is NOT able to cut the target-strand and the non-target-strand when they are in the double strand DNA form. The mutant is not able to cut the target-strand alone as single strand DNA (FIG. 4A, B). The mutant is able to cut non-specific ssDNA although with a low activity. The entire Lid region seems to be important for the activity of the protein mutation in this region will induce changes in the activity of the protein. Interestingly, activity of this mutant carrying a substitution of the Lid domain is preserved despite substitution of the E1006. The present inventors have surprisingly found that if the entire Lid domain, including E1006, is substituted, activity is preserved provided that the residue corresponding to position 917 of the wild type FnCpf1 is non-substituted, or at the most substituted from an aspartate (D) to a glutamate (E).

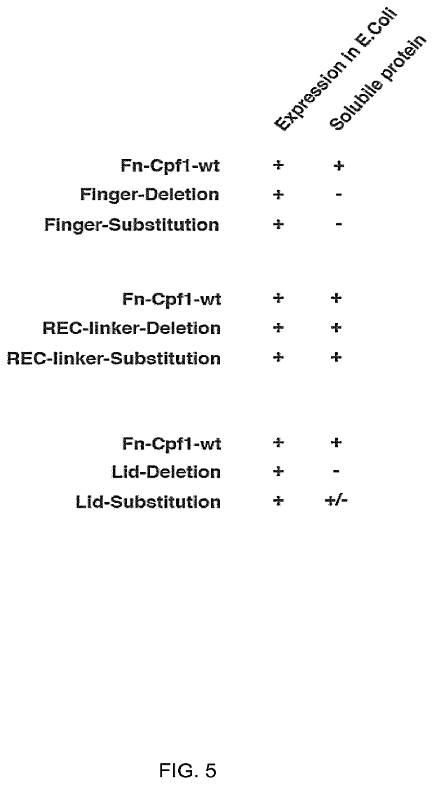

A Cpf1 mutant carrying a deletion of the entire Lid domain (SEQ ID NO: 40) is not soluble (FIG. 5).

A Cpf1 mutant carrying a deletion of the entire finger domain (SEQ ID NO: 42) is not soluble (FIG. 5).

### REFERENCES

Chen, J. S., Ma, E., Harrington, L. B., Da Costa, M., Tian, X., Palefsky, J. M., and Doudna, J. A. (2018). CRISPR-Cas12a target binding unleashes indiscriminate single-stranded DNase activity. Science. April 27; 360 (6387): 436-439.
Fonfara, I., Richter, H., Bratovic, M., Le Rhun, A., and Charpentier, E. (2016). The CRISPR-associated DNA-cleaving enzyme Cpf1 also processes precursor CRISPR RNA. Nature 532, 517-521.
Makarova, K. S., Aravind, L., Wolf, Y. I., and Koonin, E. V. (2011). Unification of Cas protein families and a simple scenario for the origin and evolution of CRISPR-Cas systems. Biol Direct 6, 38.
Makarova, K. S., and Koonin, E. V. (2015). Annotation and Classification of CRISPR-Cas Systems. Methods Mol Biol 1311, 47-75.
Mojica, F. J., Diez-Villasenor, C., Garcia-Martinez, J., and Soria, E. (2005). Intervening sequences of regularly spaced prokaryotic repeats derive from foreign genetic elements. J Mol Evol 60, 174-182.
Vestergaard, G., Garrett, R. A., and Shah, S. A. (2014). CRISPR adaptive immune systems of Archaea. RNA Biol 11, 156-167.
Zetsche, B., Gootenberg, J. S., Abudayyeh, O. O., Slaymaker, I. M., Makarova, K. S., Essletzbichler, P., Volz, S. E., Joung, J., van der Oost, J., Regev, A., et al. (2015). Cpf1 Is a Single RNA-Guided Endonuclease of a Class 2 CRISPR-Cas System. Cell 163, 759-771.
Stella, S; Mesa, P; Thomsen, J; Paul, B; Alcón, P; Jensen, S B; Saligram, B; Moses, M E; Hatzakis, N S.; Montoya, G. (2018) Conformational Activation Promotes CRISPR-Cas12a Catalysis and Resetting of the Endonuclease Activity. Cell, 175 (7) 1856-1871.

### Sequence Overview

| | |
|---|---|
| SEQ ID NO: 1 | FnCpf1 DNA sequence |
| SEQ ID NO: 2 | FnCpf1 amino acid sequence |
| SEQ ID NO: 3 | Double mutant - Nickase, Cpf1 K1013G-R1014G |
| SEQ ID NO: 4 | Double mutant - Nonspecific endonuclease, Cpf1 |
| | R918G-K1013G |
| SEQ ID NO: 5 | crRNA constant region |
| SEQ ID NO: 6 | t-strand |
| SEQ ID NO: 7 | nt-strand |
| SEQ ID NO: 8 | t-strand |
| SEQ ID NO: 9 | nt-strand |
| SEQ ID NO: 10 | crRNA |
| SEQ ID NO: 11 | REC domain of FnCpf1, DNA sequence |
| SEQ ID NO: 12 | REC domain of FnCpf1, amino acid sequence |
| SEQ ID NO: 13 | mutated REC domain of FnCpf1, DNA sequence |
| SEQ ID NO: 14 | mutated REC domain of FnCpf1, amino acid sequence |
| SEQ ID NO: 15 | Lid domain of FnCpf1, DNA sequence |
| SEQ ID NO: 16 | Lid domain of FnCpf1, amino acid sequence |
| SEQ ID NO: 17 | mutated Lid domain of FnCpf1, DNA sequence |
| SEQ ID NO: 18 | mutated Lid domain of FnCpf1, amino acid sequence |
| SEQ ID NO: 19 | finger domain of FnCpf1, DNA sequence |
| SEQ ID NO: 20 | finger domain of FnCpf1, amino acid sequence |
| SEQ ID NO: 21 | mutated finger domain of FnCpf1, DNA sequence |
| SEQ ID NO: 22 | mutated finger domain of FnCpf1, amino acid |
| | sequence |
| SEQ ID NO: 23 | labelled non-specific ssDNA |
| SEQ ID NO: 24 | Cpf1 of Acidaminococcus sp. BV3L6 |
| SEQ ID NO: 25 | Cpf1 of Lachnospiraceae bacterium COE1 |
| SEQ ID NO: 26 | Cpf1 of Succiniclasticum ruminis |
| SEQ ID NO: 27 | Cpf1 of Butyrivibrio fibrisolvens |
| SEQ ID NO: 28 | non-specific ssDNA |
| SEQ ID NO: 29 | FnCpf1 Q1025G mutant |
| SEQ ID NO: 30 | FnCpf1 Q1025G mutant |
| SEQ ID NO: 31 | FnCpf1 Q1025G E1028G mutant |
| SEQ ID NO: 32 | FnCpf1 Q1025G E1028G mutant |
| SEQ ID NO: 33 | Rec-deletion mutant FnCpf1 |
| SEQ ID NO: 34 | Rec-deletion mutant FnCpf1 |
| SEQ ID NO: 35 | Rec-substitution FnCpf1 mutant |
| SEQ ID NO: 36 | Rec-substitution FnCpf1 mutant |
| SEQ ID NO: 37 | LID-substitution FnCpf1 mutant |
| SEQ ID NO: 38 | LID-substitution FnCpf1 mutant |
| SEQ ID NO: 39 | LID-Deletion FnCpf1 mutant |
| SEQ ID NO: 40 | LID-Deletion FnCpf1 mutant |
| SEQ ID NO: 41 | Finger-deletion FnCpf1 mutant |
| SEQ ID NO: 42 | Finger-deletion FnCpf1 mutant |
| SEQ ID NO: 43 | Cpf1 of Candidatus Methanoplasma termitum |
| SEQ ID NO: 44 | Cpf1 of uncultured Clostridium sp. |

1. A mutant Cpf1 endonuclease or orthologue thereof comprising a polypeptide sequence having at least 95% sequence identity to:
i) the sequence corresponding to residues 1 to 323, 337 to 1005, and 1019 to 1329 of SEQ ID NO: 2, wherein said polypeptide sequence further comprises:
a. at least two amino acid mutations in the REC domain (residues 324 to 336; SEQ ID NO: 16) compared to SEQ ID NO: 2, wherein each mutation independently is an amino acid substitution or deletion; and/or
b. at least two amino acid substitutions in the Lid domain (residues 1006 to 1018; SEQ ID NO: 20) compared to SEQ ID NO: 2, wherein at least one of the residues at positions 917 and 1006 is a glutamic acid (E) or an aspartic acid (D);
and/or
ii) SEQ ID NO: 2, wherein said polypeptide sequence comprises at least two amino acid substitutions in two positions independently selected from the positions corresponding to residues 918, 1013, 1014, 1025 and 1028 of SEQ ID NO: 2.

2-39. (canceled)

40. A polynucleotide encoding the mutant Cpf1 endonuclease or orthologue thereof according to claim 1.

41. (canceled)

42. A recombinant vector comprising a polynucleotide according to claim 40.

43-46. (canceled)

47. A cell capable of expressing the mutant Cpf1 or orthologue thereof according to claim 1.

48. A system for expression of a crRNA-Cpf1 complex comprising
a. a polynucleotide according to claim 40;
b. a polynucleotide or a recombinant vector comprising a polynucleotide encoding a guide RNA (crRNA), optionally operably linked to a promoter; and/or
c. a cell for expression of the polynucleotide or the recombinant vector of a. and b. above.

49-50. (canceled)

51. A method for introducing a single strand break in a first target nucleic acid, wherein the method comprises:
a. contacting the Cpf1 endonuclease or orthologue thereof of claim 1 with a guide RNA (crRNA), thereby obtaining a crRNA-Cpf1 complex capable of recognising a second target nucleic acid, the second target nucleic acid comprising a protospacer adjacent motif (PAM); and
b. contacting the crRNA-Cpf1 complex with the first target nucleic acid;
whereby a single strand break is made in the first target sequence.

52-87. (canceled)

88. A method of introducing a single strand break in a first target nucleic acid, comprising the steps of:
a. designing a guide-RNA (crRNA) capable of recognising a second target nucleic acid comprising a protospacer adjacent motif (PAM);
b. contacting the crRNA of step a. with a Cpf1 endonuclease or orthologue thereof, thereby obtaining a crRNA-Cpf1 complex capable of binding to said second target nucleic acid, and
c. contacting the crRNA and the Cpf1 with said first target nucleic acid, thereby introducing one or more single strand breaks in the first target nucleic acid.

89-94. (canceled)

95. An in vitro method of introducing a site-specific, double-stranded break at a second target nucleic acid in a mammalian cell, the method comprising introducing into the mammalian cell a crRNA-Cpf1 complex, wherein the Cpf1 is a mutant Cpf1 endonuclease or orthologue according to claim 1, and wherein the crRNA is specific for the second target nucleic acid.

96. A method for detection of a second target nucleic acid in a sample, the method comprising:
a. Providing a crRNA-Cpf1 complex, wherein the Cpf1 is a Cpf1 endonuclease or orthologue thereof according to claim 1, and wherein the crRNA is specific for the second target nucleic acid;
b. Providing a labelled ssDNA, wherein the ssDNA is labelled with at least one set of interactive labels comprising at least one dye and at least one quencher;
c. Contacting the crRNA-Cpf1 complex and the ssDNA with the sample, wherein the sample comprises at least one second target nucleic acid; and
d. Detecting cleavage of the ssDNA by detecting a fluorescent signal from the fluorophore,
thereby detecting the presence of the second target nucleic acid in the sample, wherein step c. optionally comprises activation of the crRNA-Cpf1 complex.

97-105. (canceled)

106. An in vitro method for diagnosis of an infectious disease in a subject, the method comprising:
a. Providing a crRNA-Cpf1 complex, wherein the Cpf1 is a Cpf1 endonuclease or orthologue thereof according to claim 1, and wherein the crRNA is specific for a second target nucleic acid;
b. Providing a labelled ssDNA, wherein the ssDNA is labelled with at least one set of interactive labels comprising at least one dye and at least one quencher;
c. Providing a sample from the subject, wherein said sample comprises or is suspected of comprising the second target nucleic acid; and
d. Determining the level and/or concentration of the second target nucleic acid as defined in any one of the preceding claims,
wherein the second target nucleic acid is a nucleic acid of the genome of an infectious agent causing the disease or a fragment thereof,
thereby diagnosing an infectious disease in a subject.

107-114. (canceled)

115. A mutant Cpf1 endonuclease comprising a polypeptide sequence that comprises a deletion or a substitution in a Lid domain sequence, a finger domain sequence, or a REC domain sequence, wherein the mutant Cpf1 endonuclease exhibits non-specific single-stranded DNase activity, and exhibits reduced endonuclease activity on target DNA compared to said endonuclease activity of wild type Cpf1.

116. The mutant Cpf1 endonuclease of claim 115, wherein the polypeptide sequence comprises at least one amino acid substitution or deletion in the REC domain sequence; or at least two amino acid substitutions or deletions in the REC domain sequence.

117. The mutant Cpf1 endonuclease of claim 116, wherein the REC domain sequence of the wild type Cpf1 corresponds to the sequence at positions 324-336 of SEQ ID NO: 2; positions 305-317 of SEQ ID NO: 24; or positions 283-295 of SEQ ID NO: 25, and wherein the polypeptide sequence of the mutant Cpf1 endonuclease comprises a deletion of 13 contiguous amino acids at positions 324-336 of SEQ ID NO: 2; positions 305-317 of SEQ ID NO: 24; or positions 283-295 of SEQ ID NO: 25.

118. A mutant Cpf1 endonuclease comprising a polypeptide sequence that comprises a substitution or deletion of the amino acid residues corresponding to positions 918, 1013, 1014, 1025 and/or 1028 of SEQ ID NO: 2; 909, 1000, 1001, 1013 and/or 1016 of SEQ ID NO: 24; or positions 843, 943, 944, 955 and/or 958 of SEQ ID NO: 25, wherein the mutant Cpf1 endonuclease exhibits endonuclease activity upon a target dsDNA sequence to cleave both strands of the target dsDNA, and the mutant Cpf1 endonuclease exhibits reduced non-specific single-stranded DNase activity compared to wild type Cpf1.

119. The mutant Cpf1 endonuclease of claim 118, wherein the mutant Cpf1 comprises a substitution of the amino acid residues corresponding to positions 1000 and 1001 of SEQ ID NO: 24, optionally to glycine.

120. A mutant Cpf1 endonuclease comprising a polypeptide sequence having at least 95% identity to the sequence of SEQ ID NO: 24, wherein the polypeptide comprises at least two amino acid substitutions in the Lid domain sequence corresponding to positions 993-1005 of the amino acid sequence of SEQ ID NO: 24, optionally wherein the Lid domain sequence comprises an amino acid substitution at position 1000 and position 1001.

121. A method of cleaving a target nucleic acid, the method comprising contacting the target nucleic acid with the mutant Cpf1 endonuclease of claim 115 and a gRNA, wherein the target nucleic acid is cleaved by the mutant Cpf1 endonuclease.

122. A method of detecting a target nucleic acid, the method comprising contacting the target nucleic acid with the mutant Cpf1 endonuclease of claim 115 and a gRNA and detecting the target nucleic acid.
